(19) United States
(12) Patent Application Publication
(10) Pub. No.: US 2026/0345501 A1
(43) Pub. Date: Nov. 5, 2026

(54) ORAL FORMULATIONS WITH INCREASED UPTAKE

(71) Applicant: Brown University, Providence, RI (US)

(72) Inventors: Edith MATHIOWITZ, Providence, RI (US); Aharon AZAGURY, Providence, RI (US); Cameron BAPTISTA, Providence, RI (US)

(21) Appl. No.: 19/735,568

(22) Filed: Jul. 9, 2026

Related U.S. Application Data
(63) Continuation-in-part of application No. 17/265,751, filed on Aug. 5, 2019.
(60) Provisional application No. 62/714,454, filed on Aug. 3, 2018.
(60) Provisional application No. 62/787,186, filed on Dec. 31, 2018.

(51) Int. Cl. A61K 9/48; A61K 38/26

(52) U.S. Cl. CPC A61K 9/4816; A61K 9/4891; A61K 38/26

(57) ABSTRACT

Described are polymeric particles containing polymers and formulations containing these polymeric particles. The polymeric particles are effectively absorbed by intestinal mucosa and/or GI tissue, and show increased systemic uptake following oral administration as a result of their negative zeta potentials in DI water. The polymers contain a moiety that imparts a negative charge in DI water to the polymers. Optionally, the polymeric particles contain an anionic surfactant, lipid(s), peptide(s), salt(s), amino acids, induced electrons in appropriate quantities to induce the desired negative charge or/and to further enhance GI absorption and/or systemic uptake. The polymeric particles can be used to deliver any therapeutic, diagnostic, and/or prophylactic agent suitable for encapsulation.

### CROSS-REFERENCE TO RELATED APPLICATIONS

This application is a continuation-in-part of co-pending application Ser. No. 17/265,751, filed Feb. 3, 2021, which is a National Phase application under 35 U.S.C. § 371 of PCT/US2019/045152 filed on Aug. 5, 2019, which claims benefit and priority to U.S. Application No. 62/714,454, filed Aug. 3, 2018, and U.S. Application No. 62/787,186, filed Dec. 31, 2018, the disclosures of which are incorporated herein by reference.

### REFERENCE TO THE SEQUENCE LISTING

The Sequence Listing submitted as an XML file named “BU_2594_CIP_US.xml” created on Jun. 23, 2026, and having a size of 13,109 bytes is hereby incorporated by reference pursuant to 37 C.F.R. § 1.834(c)(1).

### FIELD OF THE INVENTION

This invention is generally in the area of systems for delivery of therapeutic, diagnostic and/or prophylactic agents, particularly the delivery of these agents via the gastrointestinal tract by oral administration.

### BACKGROUND OF THE INVENTION

The delivery of active agents, such as therapeutic, prophylactic, and/or diagnostic agents, takes a variety of forms, depending on the agent to be delivered and the administration route. A preferred route of administration is administration via oral passages for subsequent absorption by or across the intestinal mucosa into systemic circulation of the varied conditions of the gastrointestinal (GI) tract, e.g. changes in pH along the GI tract, exposure to different degrading enzymes, and the presence of the intestinal mucosa, some agents are not suited for oral administration, as they are degraded before penetrating the GI tract at these conditions and/or do not penetrate well into the GI tract and achieving systemic circulation. If penetration could be enhanced this approach could be used for local delivery to the tissue or any other systemic organ.

Some proposed oral delivery systems involve encapsulating the active agent to be delivered. The degree of interaction between encapsulating material, such as a polymeric particle, and intestinal mucosa can play a role in the efficiency of absorption of orally delivered active agents. However, the mucus lining of GI tract successfully entraps and eliminates the majority of encapsulating materials, making it hard to achieve effective amounts of these agents systemically or in some cases the intestinal epithelial tissue (i.e., local delivery). Substantial effort has been dedicated to the development of oral delivery systems based on polymeric particles. However, most of the introduced polymeric particles are entrapped and eliminated by the protective mucosal lining of the GI tract, significantly reducing the efficiency of such delivery systems.

Based on past studies, it was generally understood that for particles including polymeric particles to be efficient in mucus penetration, neutral charge is preferable (Cone, Advanced Drug Delivery Reviews 61.2 (2009): 75-85). Numerous studies indicated that coating of negatively charged particle with neutral PEG increases its diffusion and subsequent uptake through GI tract (Cone, Advanced Drug Delivery Reviews 61.2 (2009): 75-85; Lai, et al., Proceedings of the National Academy of Sciences of the United States of America 104.5 (2007): 1482-7; Griffiths, et al., European journal of pharmaceutics and biopharmaceutics: official journal of Arbeitsgemeinschaft fur Pharmazeutische Verfahrenstechnik e.V 97.Pt A (2015): 218-22; Abdulkarim, et al., European journal of pharmaceutics and biopharmaceutics: official journal of Arbeitsgemeinschaft fur Pharmazeutische Verfahrenstechnik e.V 97.Pt A (2015): 230-8). However, prior studies with PEGylated particles did not investigate systemic uptake. Positively charged particles might have too strong interactions with negatively charged mucin, entrapping them within the mucosal mesh, as was illustrated with chitosan spheres (Kas, J. Microencapsulation 14.6 (1997): 689-711). On the other hand, highly negatively charged particles might have repulsive forces with negatively charged mucin and biological membranes, which are generally negatively charged (e.g., usually about −200 mV), thus reducing the chance of mucus penetration by simply getting cleared from the body.

Although numerous studies have attempted to tackle the problem of poor mucosal penetration, little is known about the relationship(s) between the encapsulating material's physicochemical properties such as size, composition, surface charge (zeta potential), and surface chemistry, and their interaction with mucin, which is the main component of mucus.

Accordingly, there is a unmet need for the development of improved oral delivery systems.

Therefore, it is an object of the invention to develop oral delivery compositions or systems with improved properties.

It is another object of the invention to develop oral delivery compositions or systems with improved properties, after oral administration of the delivery systems.

It is another object of the invention to develop methods for increasing uptake or bioavailability of an active agent following oral administration.

### SUMMARY OF THE INVENTION

Described are particles including polymeric particles with negative zeta potentials, as determined in aqueous solution (specifically DI water), and formulations containing these polymeric particles. The oral formulations and systems described herein have improved properties for oral drug delivery, such as negative zeta potential, increased systemic uptake into blood, and/or increased local GI uptake. The polymeric particles are absorbed by intestinal mucosa and/or tissue, and show increased systemic uptake following oral administration. Without being bound by theory, it is believed that a low negative zeta potential in the presence of mucins (optionally, in mucin solution) coupled with an appreciable bioadhesivity, work together to enhance systemic uptake by increasing the diffusivity of the polymeric particles in the GI mucosa, while providing sufficient bioadhesion to prevent their clearance. However, the low negative zeta potential in the presence of mucins predominates in affording enhanced systemic uptake.

Preferred polymeric particles are those with a zeta potential in DI water between −20 mV and −70 mV, particularly between −40 mV and −60 mV. Preferably, the polymeric particles have a bioadhesion force of 500 mN/cm² or greater. Based upon whether delivery is local in the GI tract or systemic, the particles can have different sub-ranges of size. For example, the diameters of the polymeric particles for local delivery in the GI tract are generally between 900 nm and 2000 nm; diameters for polymeric particles for systemic delivery are generally between 100 nm and 800 nm. Generally, the polymers contain a moiety that imparts a negative charge to the polymeric particles in water. Typically, the moiety is present on the surface of the polymeric particles. The negative charge could also be an integral part of the polymer that encapsulates the agent to be delivered. The negative charge could also be obtained by surface modification, such as through physical adsorption of a moiety that imparts the negative charge.

Preferred polymers are hydrophobic, biodegradable and biocompatible polymers that have preferred a molecular weight between 2 kDa and 10 kDa to impart a high charge density to the surface of the polymeric particles. However, higher molecular weights are also useful as long as they have the appropriate charge. The property of negative charge can be determined in deionized (DI) water for all particles, including polymeric particles. Preferred polymers are poly(lactic acid) and poly(fumaric-co-sebacic acid)).

The polymeric particles also contain therapeutic agents, prophylactic agents, and/or diagnostic agents. Preferably, the therapeutic agents, prophylactic agents, and/or diagnostic agents are encapsulated in the polymeric particles.

Optionally, the polymeric particles contain an anionic or zwitterionic surfactant/chemical agent in small quantities to further enhance GI absorption and/or systemic uptake. The polymeric particles show enhanced uptake into systemic circulation of between 10% and 80%, between 10% and 70%, between 20% and 75%, between 20% and 70%, between 30% and 70%, or between 30% and 60% in a mammal, as measured using Fourier Transform Infrared (FTIR) spectroscopy.

Also described are methods of making and using the polymeric particles.

### BRIEF DESCRIPTION OF THE DRAWINGS

FIG. 1 is a column graph showing the zeta potential measurements (surface charge) of various polymeric particles in DI water (left, blue bars) and in 0.1% w/v mucin solution (right, red bars). It demonstrates the effect of mucin on the surface charge of these particles. For each type of polymer, data from the tests in water are shown on the left side, and data from the tests in mucin are shown on the right side. Results represent an average of at least three repartitions and their respective standard deviation. * p<0.1; ** p<0.05; *** p<0.005, **** p<0.0005.

FIGS. 2A-2D are point series graphs showing the relationships between the zeta potentials (surface charge) of the tested polymeric particles and either their bioadhesion force or bioadhesion work. FIG. 2A shows the zeta potential (surface charge) in 0.1% w/v mucin of various polymeric particles and their respective bioadhesion force. FIG. 2B shows the zeta potential (surface charge) in 0.1% w/v mucin of various polymeric particles and their respective bioadhesion work. FIG. 2C shows the zeta potential (surface charge) in DI water of various polymeric particles and their respective bioadhesion force. FIG. 2D shows the zeta potential (surface charge) in DI water of various polymeric particles and their respective bioadhesion work.

FIGS. 3A-3C are column graphs showing absorption of polystyrene polymeric particles in the different sections of the gastrointestinal (GI) tract post in vitro experiment. FIG. 3A shows the absorption of polystyrene polymeric particles in the duodenum, jejunum, and ileum and their respective mucus layer post in vitro experiment (absorption in mucus on the left, absorption in duodenum/jejunum/ileum in the middle, and total absorption on the right). FIG. 3B shows the absorption (%) of “Big” (1541±151 nm, right striped columns) and “Small” (310±100 nm, left solid columns) polymeric polystyrene particles in the ileum of a rat loose mucus (left), ileum tissue (middle), and total absorption (right) post in vitro experiment. FIG. 3C shows the absorption (%) of “Big” (1541±151 nm, right striped columns) and “Small” (310±100 nm, left solid columns) polymeric polystyrene particles in the duodenum of a rat loose mucus (left), duodenum (middle), and total absorption (right) post in vivo experiment. A total of 15 mg of PS particles suspended in 200 μL of phosphate-buffered saline (PBS) were administered via isolated loop experiment.

FIGS. 4A-4H are line graphs showing calibration curves for PLA (FIGS. 4A-4G) or detection of PLA particles in the blood and GI tissue of rats, and FTIR spectra of pure PLA, unexposed dry serum, and 5-h post isolated loop experiment dry serum (FIG. 411). FIG. 4A represents the calibration curve for PLA in rat's ileum tissue (local delivery) using Fourier Transform Infrared Spectroscopy (FTIR) measurements. The calibration curve represents the correlation between the specific peak ratio of tissue (1650 cm⁻¹) peak to PLA (1185 cm⁻¹) peak. This peak ratio is correlating with PLA concentration in dry GI tissues. FIGS. 4B, 4C, 4D, 4E, 4F, and 4G are line graphs depicting the calibration curves involving the detection of PLA in the blood serum using (FTIR). These (FIGS. 4B-4G) calibration curves represent the correlation between the tested pure PLA peaks (at 1750, 1188, and 1084 cm⁻¹) and the subtracted and divided specific serum peak at 1650 cm⁻¹. FIG. 4B shows the calibration curve for PLA in rat's serum (systemic uptake). The calibration curve represents the correlation between the specific peak ratio of PLA (1750 cm⁻¹) peak to serum (1650 cm⁻¹) peak after subtraction of the control peak ratio of these wavelengths. This peak ratio is correlating with PLA concentration in dry serum. PLA calibration curve was generated using the liquid method and it represents the average of at least three repetitions with their respective standard deviation. FIG. 4C shows the calibration curve for PLA in rat's serum (systemic uptake). The calibration curve represents the correlation between the specific peak ratio of PLA (1084 cm⁻¹) peak to serum (1650 cm⁻¹) peak after subtraction of the control peak ratio of these wavelengths. This peak ratio is correlating with PLA concentration in dry serum. PLA calibration curve was generated using the liquid method and it represents the average of at least three repetitions with their respective standard deviation. FIG. 4D shows the calibration curve for PLA in rat's serum (systemic uptake). The calibration curve represents the correlation between the specific peak ratio of PLA (1188 cm⁻¹) peak to serum (1650 cm⁻¹) peak after subtraction of the control peak ratio of these wavelengths. This peak ratio is correlating with PLA concentration in dry serum. PLA calibration curve was generated using the liquid method and it represents the average of at least three repetitions with their respective standard deviation. FIG. 4E shows the calibration curve for PLA in rat's serum (systemic uptake). The calibration curve represents the correlation between the specific peak ratio of PLA (1750 cm⁻¹) peak to serum (1650 cm⁻¹) peak after dividing by the control peak ratio of these wavelengths. This peak ratio is correlating with PLA concentration in dry serum. PLA calibration curve was generated using the liquid method and it represents the average of at least three repetitions with their respective standard deviation. FIG. 4F shows the calibration curve for PLA in rat's serum (systemic uptake). The calibration curve represents the correlation between the specific peak ratio of PLA (1084 cm⁻¹) peak to serum (1650 cm⁻¹) peak after dividing by the control peak ratio of these wavelengths. This peak ratio is correlating with PLA concentration in dry serum. PLA calibration curve was generated using the liquid method and it represents the average of at least three repetitions with their respective standard deviation. FIG. 4G shows the calibration curve for PLA in rat's serum (systemic uptake). The calibration curve represents the correlation between the specific peak ratio of PLA (1188 cm⁻¹) peak to serum (1650 cm⁻¹) peak after dividing the control peak ratio of these wavelengths. This peak ratio is correlating with PLA concentration in dry serum. PLA calibration curve was generated using the liquid method and it represents the average of at least three repetitions with their respective standard deviation. FIG. 4H shows an example, in one rat, of the detection of PLA particles in the blood of rats by measuring their FTIR spectrographs. FIG. 4H presents FTIR interferograms denoted pure PLA (i.e., polymer), 5-hour sample (rat's dry serum sample 5 hours post in vivo isolated loop exposure to 110 mg of PLA in 1 mL of PBS), and 0-hour control (i.e., pure dry serum sample). This specific example exhibits the peaks detected from FIG. 4H and was used in the examples described below in order to calculate the systemic (in serum) uptake of PLA particles. The peaks obtained in FIG. 4H could be used in any of FIG. 4B-G depicting the different possible calibration curves of PLA in dry serum. For example, the peaks detected in FIG. 4H were used to calculate the peak ratio of PLA (1750, 1188, or 1084 cm⁻¹) to the serum (1650 cm⁻¹) which is used in FIGS. 4B-4G calibration curves to determine the systemic uptake (in the blood) of PLA (see full calculation in Example 4 below). Generally, peak ratios were preferred as they serve as an internal control for the tissue/serum. FIG. 4H the x-axis shows wavenumber (cm⁻¹) ranging from 4000 cm⁻¹ to 400 cm⁻¹. The y-axis shows absorbance on a scale from 0 to 2.4. Actual measured values ranged between 0 and 2.0, inclusive.

### DETAILED DESCRIPTION OF THE INVENTION

### I. Definitions

“About,” as relates to numerical values, refers to a value that is ±10% of the specified value.

“Bioadhesion” and related terms refer to the phenomenon where a substance (e.g. a polymer or particle containing such a polymer) adheres to a biological surface, such as an epithelial surface, or mucus preferably on an epithelial surface, or both. Preferably, adhesion occurs in an aqueous environment. Mucoadhesion is a more specific form of bioadhesion that refers to the interaction of a substance and the mucosal tissue. Bioadhesion and mucadhesion are used interchangeably. Bioadhesion can also be quantitated in relative terms, such as, but not limited to, a spectrum of bioadhesiveness within a group of substances, such as polymers and/or polymeric particles. In some forms wherein two or more polymers or polymeric particles are being discussed, the terms “bioadhesion” and “mucoadhesion” can be defined based on a polymer's or polymeric particle's relative bioadhesiveness when compared to another, more bioadhesive polymer or polymeric particle, respectively. Bioadhesion can be measured as described in Chickering and Mathiowitz, Journal of Controlled Release (1995), 34: 251-261; U.S. Pat. No. 6,197,346 to Mathiowitz, et al.; and U.S. Pat. No. 6,235,313 to Mathiowitz, et al., the contents of which are hereby incorporated by reference.

“Negatively charged moiety,” refers to a functional group that imparts a negative charge to a molecule in a specific medium (such as an aqueous medium), particle, or other chemical groups to which it is attached, covalently or non-covalently. Preferably, the negatively charged moiety is covalently attached to the molecule, particle, or other chemical groups. Examples of negatively charged moieties include acidic groups, electrons (induction) and anionic groups.

“Acidic group” refers to a functional group that is capable of donating protons or accepting a lone pair of electrons.

“Anionic group” refers to a functional group that is the salt of an acidic group. An anionic group can be formed from the deprotonation of an acidic group, or as in the case of boronic acid, from reacting with a Lewis base such as water, hydroxyl group, thiol group, or amino group, or by water solvating a salt, such as Cl⁻ in NaCl. In solution, the acidic group and the anionic group generally exist in equilibrium, with the relative concentration of either group dependent on the pH of the solution.

“Hydrophobic” refers to the property of lacking affinity for or repelling water. For example, the more hydrophobic a polymer (or polymer segment), the more that polymer (or polymer segment) tends to not dissolve in, not mix with, or not be wetted by water. Hydrophobicity can be quantified by measuring its partition coefficient between water (or a buffered aqueous solution) and a water-immiscible organic solvent, such as octanol (then referred to as Log Kow or Log P), methylene chloride, or methyl tert-butyl ether. If after equilibration a greater concentration of the compound is attained in the organic solvent than in water, the compound is considered hydrophobic. For example, if the organic solvent is octanol, then a positive log P value above 1 indicates that the compound is hydrophobic. Whether a material is hydrophobic can also be determined via contact angle. For example, if a material is applied to a surface, such as glass, and forms a contact angle with water, which is greater than the contact angle of water on a surface of glass without the material, the material is hydrophobic. Hydrophobicity can also be quantitated in relative terms, such as, but not limited to, a spectrum of hydrophobicity within a group of polymers or polymer segments. In some forms wherein two or more polymers are being discussed, the term “hydrophobic polymer” can be defined based on the polymer's relative hydrophobicity when compared to another, less hydrophobic polymer.

“Small molecule” generally refers to an organic molecule that is less than about 2000 Da in molecular weight, less than about 1500 Da, less than about 1000 Da, less than about 800 Da, or less than about 500 Da. In some forms, small molecules are non-polymeric and/or non-oligomeric.

As used herein, the terms “effective amount” and “therapeutically effective amount” mean a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

The term “room temperature” refers to a temperature between about 288 K and about 303 K, such as 298 K.

### II. Compositions

Polymeric particles and formulations containing these polymeric particles for enhanced absorption by intestinal mucosa and/or tissue, as well as increased systemic uptake following oral administration, are described herein. The polymeric particles display controlled release properties. These improved properties can be attributed to the zeta potential and/or size of the polymeric particles, particularly the negative zeta potentials in DI water possessed by these particles. As shown by the following Examples, polymeric particles with low negative zeta potentials in water, displayed enhanced GI absorption and/or systemic uptake.

The polymeric particles may contain one or more polymers that are negatively charged or contain a moiety that has a negative electrostatic potential. In some forms, the polymers contain a moiety that imparts a negative charge or a negative zeta potential to the polymers. Preferably, these moieties are present on the surface of the polymeric particles, such that the polymeric particles display negative zeta potentials in water.

The polymeric particles typically have a diameter between 100 nm and 5000 nm, inclusive. However, based upon whether delivery is local, such as to the GI tract, or to the systemic circulation, the particles can have different size sub-ranges within this range. Typically, the diameters of the polymeric particles for local delivery are in the range from 900 nm to 5000 nm, inclusive, such as between 900 nm and 2000 nm. Typically, the diameters of the polymeric particles for systemic delivery are in the range from 100 nm to 800 nm.

In some forms, the polymeric particles contain polymers that are not bioadhesive. In some forms, the polymeric particles contain polymers that are bioadhesive. As discussed above, the bioadhesive properties of a polymer can be defined based on its relative bioadhesivity when compared to another more bioadhesive polymer.

Preferred polymers are hydrophobic, biodegradable and biocompatible polymers that degrade rather than dissolve in an aqeuous medium. Polymer dissolution can be determined as described in Estrellas, et al., Colloids and Surfaces B: Biointerfaces 173 (2019), 454-469, the contents of which are hereby incorporated by reference. In some forms, polymer dissolution can be determined as a function of time, i.e., rate of dissolution, at a given pH. For example, polymers can stay intact for a certain time period (e.g. about one hour) and pH (e.g. between 6 and 7), and subsequently dissolve. Preferably, these preferred polymers (i.e., hydrophobic, biodegradable and biocompatible polymers) have a molecular weight between 2 kDa and 20 kDa, preferably about 2 kDa, about 2.5 kDa, about 5 kDa, about 8 kDa, 10 kDa, 15 kDa, or 20 kDa. In some forms, the polymeric particles contain a blend of a low molecular weight polymer, such as one having a molecular weight between 2 kDa and 20 kDa, between 2 kDa and 15 kDa, or between 2 kDa and 10 kDa, and high molecular weight polymer having a molecular weight higher than this range, such as between 21 kDa and 300 kDa, for example, in the range of greater than 20 kDa and up to 300 kDa, greater than 20 kDa and up to 100 kDa, between 25 kDa to 50 kDa, between 30 kDa and 100 kDa, between 100 kDa and 200 kDa, or between 200 kDa and 300 kDa. When the polymeric particles contain such a blend, the ratio of the low molecular weight polymer to the high molecular weight polymer can be between 30% wt/wt and 90% wt/wt, inclusive, such as between 30% wt/wt and 40% wt/wt, between 40% wt/wt and 50% wt/wt, between 50% wt/wt and 60% wt/wt, between 60% wt/wt and 70% wt/wt, between 70% wt/wt and 80% wt/wt, or between 80% wt/wt and 90% wt/wt. Preferred polymers are polyesters (e.g. poly(lactic acid)) and polyanhydrides (e.g. poly(fumaric-co-sebacic acid)). Preferably, the polymeric particles also contain therapeutic agents, prophylactic agents, and/or diagnostic agents encapsulated therein. Any of these agents, suitable for encapsulation can be encapsulated and delivered to the GI tract/blood stream (systemic) via the polymeric particles.

Optionally, the polymeric particles can contain additional components, such as an anionic surfactant, peptides, lipids, amino acids, salts, or a combination thereof, in small quantities to further enhance GI absorption and/or systemic uptake. Preferably, these additional components are separate entities from the therapeutic agents, prophylactic agents, and/or diagnostic agents described herein. The polymeric particles show enhanced uptake into systemic circulation, of between 10% and 70%.

### A. Polymeric Particles

The components and properties of the polymeric particles: polymer, a moiety that imparts a negative charge, size, zeta potential, GI and tissue absorption, systemic uptake, and the optional additional components, such as anionic surfactant, are further described in the ensuing sections.

Upon reaching their target locale or systemically, the polymeric particles, preferably, release the agent to be delivered in a controlled release manner. The moiety that imparts a negative charge can be bonded covalently or non-covalently to the polymers, polymeric particle surface, therapeutic agents, prophylactic agents, and/or diagnostic agents. Exemplary non-covalent bonds include, but are not limited to, electrostatic interactions, affinity interactions, metal coordination, physical adsorption, host-guest interactions, and hydrogen bonding interactions. The moiety that imparts a negative charge is considered part of the polymeric particle even when the moiety is non-covalently bonded to the polymers or surface of the polymeric particles via at least one of the non-covalent bonds described herein.

Preferably, the polymeric particles do not contain chemically bound poly(ethylene glycol) (PEG) on their surface. Preferably, the polymeric particles do not contain chemically bound PEG on their surface, at a density that imparts a near-neutral zeta potential to the particle. “Near-neutral” zeta potential can be a zeta potential between −10 mV and +10 mV, inclusive, between −7.5 mV and +7.5 mV, inclusive, or between −5 mV and +5 mV. Additionally, generally, the polymeric particles do not contain poly(butadiene-maleic anhydride-co-L-dopamine) (PBMAD) or a polymer with water solubility similar to that of PBMAD, as measured under the same conditions (e.g., pH, temperature, and pressure). The polymers can be used to (i) form the entire matrix of the polymeric particles or (ii) coat the surface of the polymeric particles. Preferably, when the polymers form the entire matrix of the polymeric particles, the particles can be manufactured using a method such as phase inversion nanoencapsulation (PIN) phenomenon, described in U.S. Patent Application Publication 2004/0070093A1 by Mathiowitz, et al., the contents of which are hereby incorporated by reference. Preferably, when the polymers form a coating on the surface of the polymeric particles, the polymeric particles can be manufactured using a method such as single step double-walled nanoencapsulation (SSDN), described in detail in Azagury, et al., Journal of Controlled Release 280, (2018), 11-19, the contents of which are incorporated herein by reference.

### (i) Polymers

Preferably, the polymers are hydrophobic, i.e., hydrophobic polymers. Preferably, the polymers do not dissolve immediately in water. Preferred polymers also include biodegradable polymers that are non-soluble in the GI tract or in a medium having a pH between 1 and 7, inclusive, over a period of time such as five minutes, 10 minutes, 15 minutes, 20 minutes, 30 minutes, 45 minutes, or one hour after contact with the GI tract or the medium. “Non-soluble” can refer to less than 2% wt/wt of the polymers dissolving during this period of time. As discussed above, the polymers can be used to (i) form the entire matrix of the polymeric particles, and/or (ii) coat the surface of the polymeric particles.

Preferably, the polymers contain or are biodegradable or bioerodible and biocompatible polymers. The biodegradable/biocompatible polymers can be homopolymers, copolymers, or a combination thereof. Biodegradable/biocompatible polymers can include one or more of the following: polyesters (poly(caprolactone); poly(hydroxy acids), such as poly(lactic acid), poly(glycolic acid), and poly(lactic acid-co-glycolic acids); polyhydroxyalkanoates, such as poly(3-hydroxybutyrate) and poly(4-hydroxybutyrate)); polyanhydrides (poly(fumaric-co-sebacic acid), polysebacic acid, polyfumaric acid); poly(orthoesters); hydrophobic polypeptides; hydrophobic polyethers, such as poly(propylene oxide); poly(phosphazenes), polyesteramides, poly(alkylene alkylates), polyether esters, polyacetals, polycyanoacrylates, polyketals, polyhydroxyvalerates, polyalkylene oxalates, polyalkylene succinates, mixtures, and copolymers thereof. In particularly preferred embodiments, the biodegradable/biocompatible polymers are hydrophobic, i.e., hydrophobic, biodegradable and biocompatible polymers.

Biodegradable and biocompatible polymers containing lactic acid units, such as poly-L-lactic acid, poly-D-lactic acid, poly-D, L-lactic acid, poly-L-lactide, poly-D-lactide, and poly-D, L-lactide, are collectively referred to herein as “PLA.” Those that contain caprolactone units, such as poly(s-caprolactone), are collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA.”

The preferred hydrophobic polymer in the polymer is poly(lactic acid), poly(fumaric-co-sebacic acid), poly(glycolic acid), or poly(lactic acid-co-glycolic acid).

In more preferred embodiments, the polymer contains a moiety that imparts a negative charge to the polymer in DI water. The moiety can be incorporated before or after the polymeric particle is formed. Preferably, the moiety is incorporated or present in the polymer before the particle is formed. Preferably, the moiety is covalently attached to the polymer. Preferably, the moiety is on the surface of the polymeric particle. The moiety can be an acidic group, an anionic group, peptide(s), amino acid(s), lipid(s), salt(s), or combinations thereof. Examples of acidic groups include, but are not limited to, carboxylic acids, protonated sulfates, protonated sulfonates, protonated phosphates, singly- or doubly protonated phosphonates, and singly- or doubly protonated hydroxamate. The corresponding salts of these acidic groups form anionic groups such as carboxylates, sulfates, sulfonates, singly- or doubly deprotonated phosphate, singly- or doubly deprotonated phosphonate, and hydroxamate. Preferred acid and anionic groups are carboxylic acids and carboxylates, respectively.

The polymer typically has a molecular weight between 1.5 kDa and 300 kDa, inclusive, 1.5 kDa and 275 kDa, inclusive, 1.5 kDa and 250 kDa, inclusive, between 1.5 kDa and 100 kDa, between 2 kDa and 80 kDa, inclusive, between 2 kDa and 50 kDa, inclusive, between 2 kDa and 30 kDa, inclusive, between 2 kDa and 20 kDa. In some forms, in designing polymeric particles that contain polymers, polymers that have a high charge density are preferred. A high charge density can be accomplished by using polymers having low molecular weights (such as between 2 kDa and 10 kDa, inclusive, or between 2 kDa and 20 kDa, inclusive), since low molecular weight polymers in the polymeric particles contain high charge density at the end groups. Accordingly, in some forms, the polymer has a molecular weight between 2 kDa and 20 kDa, inclusive, 2 kDa and 10 kDa, inclusive, preferably about 2 kDa, about 2.5 kDa, about 5 kDa, about 8 kDa, about 10 kDa, about 15 kDa, or about 20 kDa. Polymers having higher molecular weights (such as 10 kDa and 300 kDa, for example, in the range of greater than 20 kDa and up to 300 kDa, greater than 20 kDa and up to 100 kDa, between 25 kDa to 50 kDa, between 30 kDa and 100 kDa, between 100 kDa and 200 kDa, or between 200 kDa and 300 kDa) can also be used. As discussed above, polymers can be a blend of a low molecular weight polymer having a molecular weight between 2 kDa and 20 kDa and a high molecular weight polymer having a molecular weight between 21 kDa and 300 kDa. The ratio of the low molecular weight polymer to the high molecular weight polymer can be between 30% wt/wt and 90% wt/wt, inclusive, between 40% wt/wt and 90% wt/wt, inclusive, between 50% wt/wt and 90% wt/wt, inclusive, between 60% wt/wt and 90% wt/wt, inclusive, between 70% wt/wt and 90% wt/wt, inclusive, or between 80% wt/wt and 90% wt/wt, inclusive. Preferably, the polymeric particle has a zeta potential between −10 mV and −80 mV, such as between −20 mV and −70 mV, or between −40 mV and −60 mV.

### (ii) Anionic Surfactants

In some forms, the polymeric particles can include an anionic surfactant. The anionic surfactant preferably enhances gastrointestinal and/or systemic uptake of the polymeric particles. In some forms, when present, the anionic surfactant constitutes between about 0.0001% wt/wt and about 5% wt/wt, inclusive, between about 0.001% wt/wt and about 5% wt/wt, of the polymeric particles. Preferably, the anionic surfactants are present on the surface of the polymeric particles. In these forms, the anionic surfactants can be added in a solution post-polymeric particle formation, followed by washing of the polymeric particles, and retaining the anionic surfactants on the surface of the polymeric particles. The anionic surfactant can be bonded covalently or non-covalently to the polymers or polymeric particle surface. Preferably the anionic surfactant is bonded non-covalently to the polymeric particle surface via electrostatic interactions, affinity interactions, metal coordination, physical adsorption, host-guest interactions, or hydrogen bonding interactions. Preferably, the non-covalent bond is via physical adsorption.

Suitable anionic surfactants typically include those containing any of carboxylate, sulfonate, salts, amino acids, peptides, and sulfate ions. These include, but are not limited to, petroleum sulfonate, naphthalenesulfonate, olefin sulfonate, an alkyl sulfate, sulfated natural oil, sulfated fat, sulfated ester, a sulfated alkanolamide, a sulfated alkylphenol, a sulfated alkylphenol ethoxylate, laureate, lauryl ether sulfate, lauryl sulfate, decyl sulfate, octyl sulfate, a alkylbenzene sulfonate (a linear alkylbenzene sulfonate, or a branched alkylbenzene sulfonate, or a combination thereof), or a combination thereof. The combination can include anionic surfactants selected from (i) the species listed above, (ii) species listed above and species within the classes of surfactants, (iii) species within each class of surfactant, or (iv) species within different classes of surfactant.

(iii) Zeta Potential and Bioadhesivity

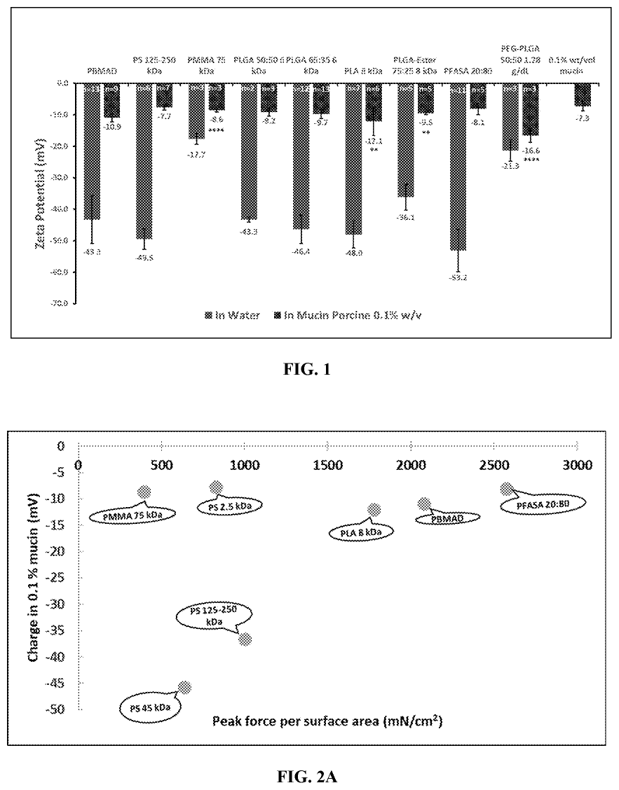

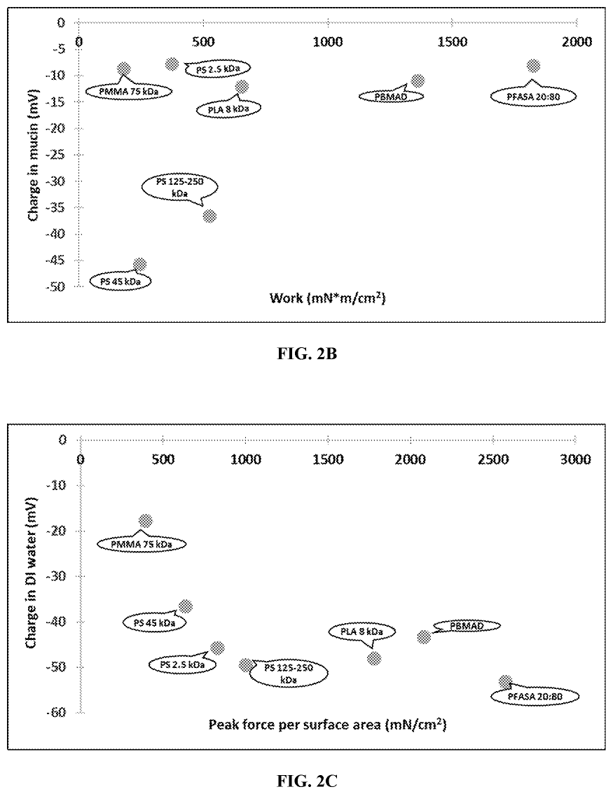

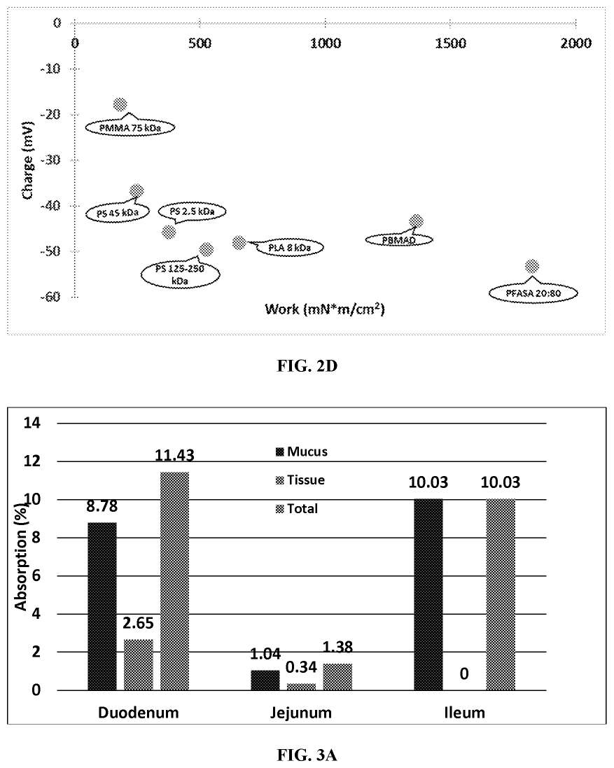

The effects of the medium/media on the zeta potential of polymeric particles were investigated by measuring the zeta potential of a series of polymeric particles both in water and in 0.1% w/v mucin in water solution, FIG. 1. As shown in FIG. 1, the polymeric particles all had negative zeta potentials, although the zeta potentials in water were significantly more negative than those in mucin. Further, the relationships between zeta potential and bioadhesion force or bioadhesion work were investigated by analyzing these parameters in water and in mucin solution, FIGS. 2A, 2B, 2C, and 2D. As shown in FIGS. 2A and 2B, these parameters (zeta potential and bioadhesion force or bioadhesion work) showed no correlation with zeta potential charge in mucin medium. Further, the relationship between zeta potential and bioadhesion force or bioadhesion work showed no correlation when studied in DI water FIGS. 2C and 2D. In preferred embodiments, the zeta potential of the polymeric particles is measured in water, preferably deionized water, at a pH of about 7.4 using a Zetasizer or similar instrument such as Zetaview, and at room temperature.

When comparing the polymeric particle zeta potentials in water to mucin, it appears that mucin has the ability to coat the particles and skew the measurement towards a less negative value (i.e. values similar to the control measurement of mucin alone, FIG. 1). This is supported by the relationship between zeta potential in the differing media and bioadhesion force or bioadhesion work, FIGS. 2A, 2B, 2C, and 2D. It is known that bioadhesive polymers can interact strongly with mucin, thus the discrepancy between the zeta potential measurements in water and mucin is seen most drastically for bioadhesive polymers (i.e., PBMAD and PFASA), while seen less drastically for non-bioadhesive polymers (i.e., PMMA and PS). Accordingly, to avoid zeta potential masking by mucin, in preferred embodiments, the zeta potential of the particles is measured in water, preferably deionized (DI) water, at a pH of about 7.4 using a Zetasizer or similar instrument such as Zetaview, and at room temperature. Deionized (DI) water typically has a pH of about 7; however, when it comes in contact with carbon dioxide, the pH of deionized water can be between 5 and 6. Accordingly, in some forms, the zeta potential of the polymeric particles can be measured in deionized water, at a pH between about 5 and about 7.4, between about 5 and about 6.5, between about 5 and about 6, or between 5 and 6, inclusive, using a Zetasizer or similar instrument (such as Zetaview), and at room temperature.

The zeta potential in DI water of the polymeric particles can be between −10 mV and −80 mV, between −15 mV and −70 mV, between −20 mV and −70 mV, between −20 mV and −60 mV, between −30 mV and −60 mV, or between −40 mV and −60 mV. In some forms, the polymeric particles have a zeta potential in the ranges described hereinbefore or after the loading of an agent to be delivered. Accordingly, the zeta potential of the particles can be determined before or after the loading of an agent to be delivered. Preferably, after loading the agent to be delivered, the particles have a zeta potential within these ranges.

In some forms, the polymers used to form the polymeric particles confer the negative zeta potential. In these forms, the polymer can be selected such that independently of the physicochemical properties of the agent to be delivered, the manufactured polymeric particles have a negative zeta potential between the ranges described above. The polymers can be those described above, such as polyesters (poly(caprolactone); poly(hydroxy acids), such as poly(lactic acid), poly(glycolic acid), and poly(lactic acid-co-glycolic acids); polyhydroxyalkanoates, such as poly(3-hydroxybutyrate) and poly(4-hydroxybutyrate)); polyanhydrides (poly(fumaric-co-sebacic acid), polysebacic acid, polyfumaric acid). Any of the agents described herein can be incorporated, given that the negative zeta potential is primarily conferred by the polymers.

In some forms, the agents to be delivered confer the negative zeta potential to the polymeric particles. Such agents typically include those that preferably have an overall negative zeta potential (e.g. negatively charged molecules) or contain moieties (e.g. aromatic rings, electron-withdrawing groups) that have negative zeta potentials in DI water. In these forms, any of the polymers described herein can be used to manufacture the polymeric particles, given that the negative zeta potential in water is primarily conferred by the agent to be delivered.

In some forms, the polymer used to manufacture the polymeric particles and the agents to be delivered both confer the negative zeta potential in water. In these forms, the polymers and agents to be delivered can be readily selected from those described herein, which confer the negative zeta potential.

Generally, polymeric particles have a negative zeta potential coupled with an appreciable bioadhesivity. These factors can work in concert to enhance systemic uptake by increasing the diffusivity of the polymeric particles in the GI mucosa, while providing sufficient bioadhesion to prevent clearance of the polymeric particles. Accordingly, in some forms, the polymeric particles have a negative zeta potential between −10 mV and −80 mV, between −15 mV and −70 mV, between −20 mV and −70 mV, between −20 mV and −60 mV, between −30 mV and −60 mV, or between −40 mV and −60 mV, and a bioadhesion force of about 500 mN/cm² (such as 480 mN/cm²), or greater. Although a low negative zeta potential and appreciable bioadhesivity can work together to provide beneficial properties, the negative zeta potential predominates in providing the beneficial properties.

### (iv) Size and Absorption

The polymeric particles can have any diameter between 100 nm and 5000 nm, inclusive, such as between 100 nm and 2000 nm, between 100 nm and 1000 nm, between 100 nm and 500 nm, between 500 nm and 1000 nm, between 1000 nm and 2000 nm, or between 1,500 nm and 2,000 nm. As a non-limiting example, the absorption of polymeric particles formed from polystyrene was investigated in vitro and in vivo in rats. The polystyrene used to form the polymeric particles optionally have carboxylate anionic groups. Nonetheless, the absorption and/or uptake can occur in the presence or absence of these carboxylate anionic groups on the particles.

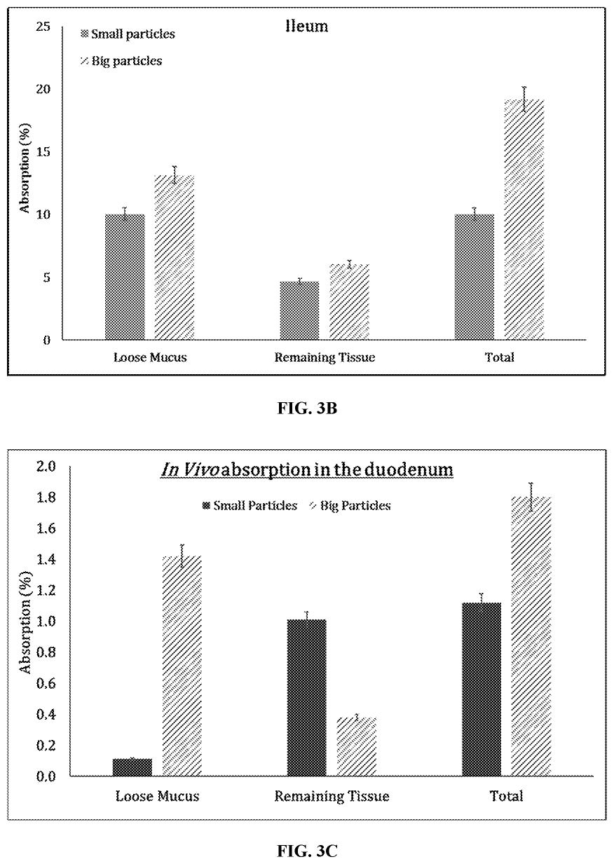

Referring to FIG. 3A, in terms of absorption between mucus and tissue, the polymeric particles showed higher absorption by duodenal, jejunal, and ileal mucus compared to their corresponding tissues, using polystyrene polymeric particles as non-limiting examples. The difference was much higher in the duodenum and ileum. The duodenum showed the highest tissue absorption, suggesting the duodenum can be a good target for polystyrene polymeric particles.

Further studies involved the effects of size on gastrointestinal absorption in vitro and in vivo, FIGS. 3B and 3C. While the polymeric particles penetrate tissue, the polymeric particles can also be taken up into systemic circulation (reaching the blood circulation). Referring to FIG. 3B, “big” (1541±151 nm) and “small” (310±100 nm) polymeric particles showed higher mucus absorption compared to tissue absorption in the duodenum in vitro. In vivo, the small polymeric particles showed higher tissue uptake compared to mucus of the ileum, FIG. 3C. Therefore, the polymeric particles can have diameters depending on the location and/or polymer type and/or type of delivery desired, namely local delivery in which an agent to be delivered predominantly remains in the GI tract or systemic delivery where the agent in the polymeric particles are absorbed into systemic circulation. Therefore, in some forms, the polymeric particles have a diameter between 100 nm and 800 nm, between 100 nm and 500 nm, between 200 nm and 400 nm, between 900 nm and 2000 nm, between about 1000 nm and 2000 nm, between 1200 nm and 2000 nm, between 1300 nm and 1800 nm. Preferably, for systemic delivery, the diameters of the polymeric particles are between 100 nm and 500 nm. Preferably, for local GI tract delivery, the diameters of the polymeric particles are between 1000 nm and 2000 nm.

### (iv) Systemic Uptake

Detecting polymeric particles containing agents in the GI tract can be used to signify local delivery agents along segments of the GI tract. Perez-Rogers, “Designing a Novel Approach to Quantify Polymeric Nanoparticle Absorption Using FTIR,” Dissertation (M.S.), Brown University, 2017, describes how to detect polymeric particles in segments of the GI tract using FTIR, the contents of which are hereby incorporated by reference. However, detecting polymeric particles containing agents in the blood can be used to signify successful absorption of both the polymeric particles and, thus, the agent to be delivered into systemic circulation. Any method known to those of skill in the art to determine polymers in samples (e.g. blood) can be used. Examples include gel permeation chromatography (GPC), high-performance liquid chromatography (HPLC), FTIR, mass spectrometry, and a combination of both (LC-MS).

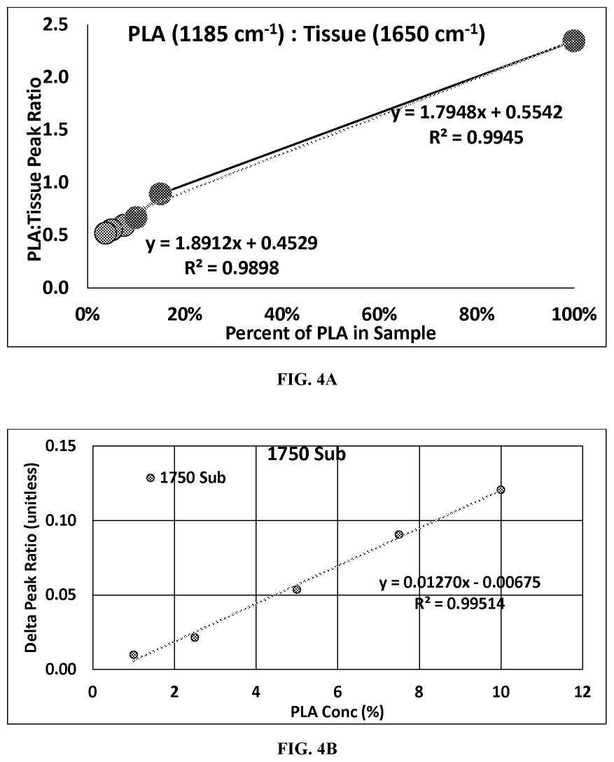

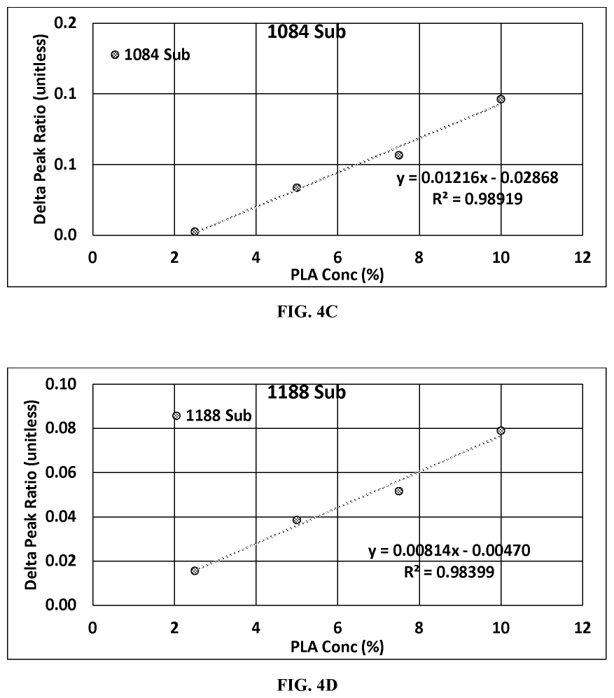

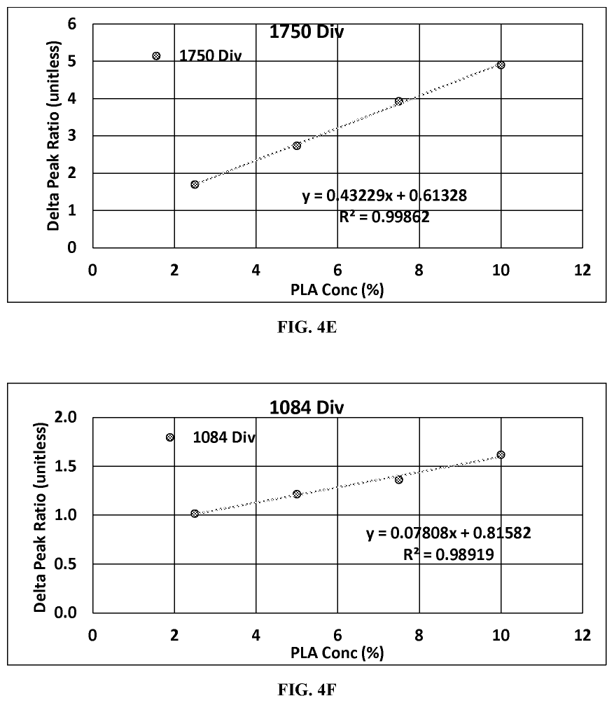

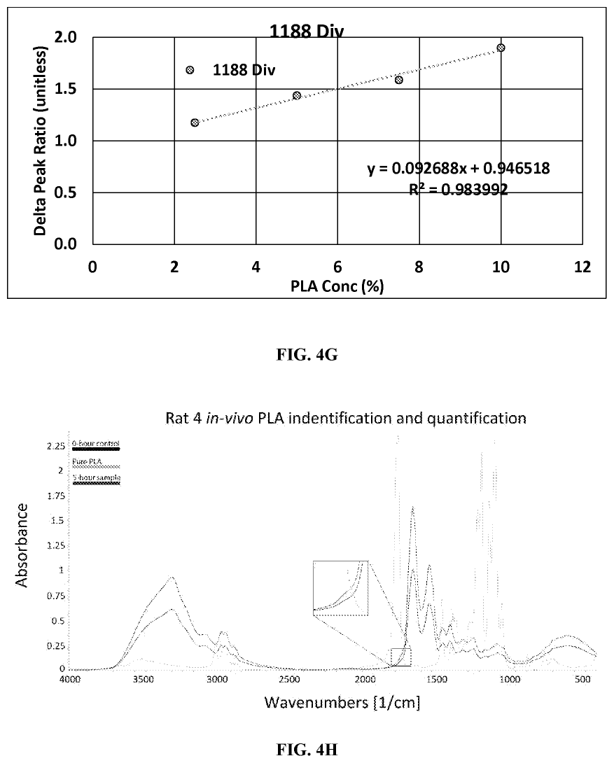

Referring to FIG. 4C, an exemplary data set is shown of a polymer PLA that was detected in blood, as shown by the peak in the magnified region. This PLA particle had negative zeta potential.

In some forms, uptake of the polymeric particles into system circulation is between 10% and 80%, between 10% and 70%, between 20% and 75%, between 20% and 70%, between 30% and 70%, or between 30% and 60%. The percentage is based on how much polymer was administered and how much of that was detected later in the blood. A strong indication for the polymer's nanoparticles uptake was the fact that after the isolated loop experiments there were no traces detected in the isolated loop washouts of the administered polymeric nanoparticles. Thus, the polymeric particles with low negative zeta potentials showed significant systemic uptake. Other polymeric particles without the low negative zeta potential described herein usually have a much lower systemic absorption or uptake <10%.

### B. Agents to be Delivered

The polymeric particles typically include agents (e.g. therapeutic agents, diagnostic agents, prophylactic agents, or a combination thereof) to be delivered to a subject.

The loading range for the agent within the polymeric particles is from about 0.01% to about 80% (agent weight/polymer weight), or from 0.01% to about 50% (wt/wt), or from about 0.01% to about 25% (wt/wt), or from about 0.01% to about 10% (wt/wt), or from about 0.1% to about 5% (wt/wt).

For large biomolecules, such as proteins and nucleic acids, typical loadings are from about0 0.01% to about 20% (wt/wt), or from about 0.01% to about 5% (wt/wt from about 0.01% to about 2.5% (wt/wt), or from about 0.01% to about 1% (wt/wt).

Compounds with a wide range of molecular weight can be encapsulated, for example, between 100 Da and 10,000 kDa. The agents to be delivered can be small molecules, proteins, polypeptides, peptides, carbohydrates, nucleic acids, antibodies, antigens, glycoproteins, lipids and combinations thereof. Preferred agents to be delivered include biologics, antibodies, antigens, and chemotherapeutics. Delivery of these agents (e.g. biologics) are not limited by their molecular weights.

Agents to be delivered contemplated for use in the polymeric particles and formulations described herein include, but are not limited to, the following categories and examples of agents and alternative forms such as alternative salt forms, free acid forms, free base forms, and hydrates:
anticancer agents (e.g. 5-fluorouracil; gemcitabine; gemcitabine hydrochloride; cytarabine; decitabine; leucovorin; acivicin, aclarubicin, acodazole hydrochloride, acronine, adozelesin, aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; dacarbazine; dactinomycin; daunorubicin hydrochloride; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; flurocitabine; fosquidone; fostriecin sodium; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimus tine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol,9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidasc; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stern cell inhibitor; stem-cell division inhibitors; stipiamide; stromelysin inhibitors; sulfinosinc; superactivc vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribinc; trimetrexate; triptorelin; tropisetron; turosteridc; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanotcrone; zeniplatin; zilascorb; and zinostatin stimalamer; analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives); antifungal agents (e.g., griseofulvin, ketoconazole, itraconizole, amphotericin B, nystatin, and candicidin); antihypertensive agents (e.g., propranolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propranolol, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine); agents useful for calcium regulation (e.g., calcitonin, and parathyroid hormone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate; steroidal compounds, hormones and hormone analogues (e.g., incretins and incretin mimetics such as GLP-1 and exenatide, androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, recombinantly produced insulin, insulin analogs, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); peptides; proteins (e.g., DNase, alginase, superoxide dismutase, and lipase); nucleic acids (e.g., sense or anti-sense nucleic acids encoding any therapeutically useful protein, including any of the proteins described herein, and siRNA); agents useful for erythropoiesis stimulation (e.g., erythropoietin); antiulcer/anti-reflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, E, K, and the like); as well as other drugs such as mitotane, halonitrosoureas, anthrocyclines, and ellipticine.

In some forms, the agent to be delivered is glucagon-like peptide-1 (GLP-1) or a truncated biologically active portion thereof or an analog thereof.

In some embodiments in which the agent to be delivered is GLP-1 or a truncated biologically active portion thereof or an analog thereof, the polymeric particle does not contain PAA (poly-adipic acid), PLGA (poly-lactic-co-glycolic acid), or PLA (poly-lactic acid) as the sole polymer forming the particle.

In some embodiments in which the agent to be delivered is GLP-1 or a truncated biologically active portion thereof or an analog thereof, the polymeric particle is not formed by phase inversion nanoencapsulation (PIN) wherein PAA (poly-adipic acid), PLGA (poly-lactic-co-glycolic acid) or PLA (poly-lactic acid) is the sole polymer used to form the particles.

In some embodiments in which the particles contain GLP-1 or a truncated biologically active portion thereof or an analog thereof, the particles do not contain PAA (poly-adipic acid).

In some embodiments in which the particles contain GLP-1 or a truncated biologically active portion thereof or an analog thereof, the particles do not contain PLGA (poly-lactic-co-glycolic acid).

In some embodiments in which the particles contain GLP-1 or a truncated biologically active portion thereof or an analog thereof, the particles do not contain PLA (poly-lactic acid).

In some embodiments in which the particles contain GLP-1, the loading of GLP-1 in the particles is not about 2.5% (wt/wt) or about 2.5% (wt/wt). In some embodiments in which the particles contain GLP-1, the loading of GLP-1 in the particles is greater than 2.5% (wt/wt) and up to about 80% (GLP-1 weight/polymer weight), or greater than 2.5% (wt/wt) and up to about 50% (wt/wt), or greater than 2.5% (wt/wt) and to about 25% (wt/wt), or greater than 2.5% (wt/wt) and up to about 10% (wt/wt), or greater than 2.5% (wt/wt) and up to about 5% (wt/wt).

In some embodiments in which the agent to be delivered is GLP-1, the polymeric particle does not contain PAA (poly-adipic acid), PLGA (poly-lactic-co-glycolic acid), or PLA (poly-lactic acid) as the sole polymer forming the particle.

In some embodiments in which the agent to be delivered is GLP-1, the polymeric particle is not formed by phase inversion nanoencapsulation (PIN) wherein PAA (poly-adipic acid), PLGA (poly-lactic-co-glycolic acid), or PLA (poly-lactic acid) is the sole polymer used to form the particles.

In some embodiments in which the particles contain GLP-1, the particles do not contain PAA (poly-adipic acid).

In some embodiments in which the particles contain GLP-1, the particles do not contain PLGA (poly-lactic-co-glycolic acid).

In some embodiments in which the particles contain GLP-1, the particles do not contain PLA (poly-lactic acid).

### (i). Glucagon-Like Peptide-1

Glucagon-like peptide-1 (GLP-1), a member of the glucagon peptide family, is a 30 amino acid long peptide hormone deriving from the tissue-specific posttranslational processing of the proglucagon gene.

Human GLP-1 (1-37) has the amino acid sequence:

(SEQ ID NO: 1)
HDEFERHAEGTFTSDVSSYLEGQAAKEFIAWLVKGRG.

The initial product GLP-1 (1-37) is susceptible to amidation and proteolytic cleavage, which gives rise to the two truncated and equipotent biologically active forms, GLP-1 (7-36) amide and GLP-1 (7-37).

Human GLP-1 (7-37) has the amino acid sequence:

(SEQ ID NO: 2)
HAEGTFTSDVSSYLEGQAAKEFIAWLVKGRG.

Human GLP-1 (7-36) has the amino acid sequence:

(SEQ ID NO: 3)
HAEGTFTSDVSSYLEGQAAKEFIAWLVKGR

Active GLP-1 contains two α-helices from amino acid position 13-20 and 24-35 (of SEQ ID NO:1) separated by a linker region.

DPP-IV cleaves the peptide bond in Ala8-Glu9 (of SEQ ID NO:1), and the resulting metabolite GLP-1(9-36)-NH2is found to have 100-fold lower binding affinity compared to the intact peptide (Manadhar and Ahn, J. Med. Chem. 2015, 58, 1020-1037). The metabolite also exhibits negligible agonistic activity (>10000-fold decrease).

Orally administered GLP-1 in a dose-escalating schedule (doses of 0.5, 1.0, 2.0, and 4.0 mg) was reported to (i) induce a rapid and dose-dependent increase in plasma drug concentrations; (ii) induce a potent effect on insulin release; and (iii) suppressed ghrelin secretion (Beglinger, et al., Clin Pharmacol Ther. 2008 October; 84(4):468-74). However, Beglinger reported bioavailabilities lower than 10%, with a mean absolute bioavailability of 4%, relative to intravenous administration of GLP-1. Further, native GLP-1 has a very short plasma half-life and is generally not suitable for therapeutic use except by continuous infusion. For example, it is possible to normalize or improve the glycemic control in type 2 diabetic patients by both intravenous and subcutaneous infusion of GLP-1 at doses of ~4 ng·kg⁻¹ min-1 or higher, however, these studies ranged from 4 to 6 hours in duration for either fasting patients or patients receiving a single meal (Larsen and Hylleberg, Diabetes Care 2001 August; 24(8): 1416-1421). Continuous 48-hour subcutaneous infusion of GLP-1 at a rate of ~4-8 ng·kg⁻¹ min also lowered fasting and postprandial glucose values in type 2 diabetic patients, and another study showed that fasting serum glucose decreased by 76.2, 53.9, 37.0 and 22.7 mg/dl for the 8.5, 5.0, 2.5 and 1.25 pmol/kg/min rGLP-1 groups, respectively, compared to a decrease of 1.1 mg/dl for placebo (Torekov., et al., Diabetes Obes Metab., 2011 July; 13(7):639-43).

### (ii). Glucagon-Like Peptide-1 Analogues

Modifying the two sites in the GLP-1 molecule susceptible to cleavage: the position 8 alanine and the position 34 lysine, can help prolong the half-life of GLP-1. These, and other chemical modifications, help in creating compounds known as GLP-1 receptor agonists, which have a longer half-life, and can be used for therapeutic purposes.

Suitable GLP-1 analogues include, for example,

exenatide (BYETTA®, BYDUREON®), liraglutide (VICTOZA®, SAXENDA®), lixisenatide (LYXUMIA®, ADLYXIN®), albiglutide (TANZEUM™), dulaglutide (TRULICITY®), semaglutide (OZEMPIC®), and taspoglutide.
a. Exenatide

Exenatide, a functional analog of GLP-1, is a synthetic version of exendin-4, a hormone found in the saliva of the Gila monster. Exenatide has the amino acid sequence:

(SEQ ID NO: 4)
HGEGTFTSDLSKQMEEEAVRLFIEWLKNGGPSSGAPPPS.

BYETTA® is an immediate-release exenatide formulated for subcutaneous (SC) injection. The recommended dosage for treating type 2 diabetes mellitus is 5 μg SC every 12 hours within 60 minutes prior to meal initially; after 1 month, may increase to 10 μg every 12 hours. BYDUREON® BCISE™ is an extended-release exenatide formulated for subcutaneous (SC) injection. The recommended dosage for treating type 2 diabetes mellitus is 2 mg subcutaneously once every 7 days (weekly), administered any time of day, with or without meals.

b. Liraglutide

Liraglutide is a long-acting, fatty acylated GLP-1 analog with prolonged action and half-life of 11-15 hours. The improved properties of liraglutide are credited to the attachment of the fatty acid palmitic acid to GLP-1 that reversibly binds to albumin and protects it from degradation and elimination and facilitates slow and consistent release. Liraglutide has the amino acid sequence HAEGTFTSDVSSYLEGQAAKEFIAWLVRGRG (SEQ ID NO:5), and has a C-16 fatty acid (palmitic acid) attached with a glutamic acid spacer on the lysine residue at position 26 of the peptide precursor (bold/italics in SEQ ID NO:5). Liraglutide is 97% homologous to native human GLP-1 with a substituted arginine for lysine at position 34.

VICTOZA® and SAXENDA® are liraglutide formulations for subcutaneous injection. A recommended dose for VICTOZA for treating type 2 diabetes mellitus is 0.6 mg SC every day for 1 week initially, then increase to 1.2 mg or 1.8 mg every day based on clinical response.

c. Lixisenatide

Lixisenatide is “des-38-proline-exendin-4 (Heloderma suspectum)-(1-39)-peptidylpenta-L-lysyl-L-lysinamide,” meaning it is derived from the first 39 amino acids in the sequence of the peptide exendin-4, omitting proline at position 38 and adding six lysine residues. The amino acid sequence of lixisenatide is

(SEQ ID NO: 6)
HGEGTFTSDLSKQMEEEAVRLFIEWLKNGGPSSGAPPSKKKKKK.

ADLYXIN® and LYXUMIA® are lixisenatide formulations for subcutaneous injection. The initial recommended dose for treating type 2 diabetes mellitus is 10 μg every day for 14 days, followed by 20 μg everyday beginning on day 15.

d. Albiglutide

Albiglutide is a dipeptidyl peptidase-4-resistant GLP-1 dimer fused to human albumin. The two GLP-1-likes domains have a single amino acid substitution relative to GLP-1(7-36). The amino acid sequence for albiglutide is:

(SEQ ID NO: 7)
HGEGTFTSDVSSYLEGQAAKEFIAWLVKGRHGEGTFTSDVSSYLEGQ
AAKEFIAWLVKGRDAHKSEVAHRFKDLGEENFKALVLIAFAQYLQQ
CPFEDHVKLVNEVTEFAKTCVADESAENCDKSLHTLFGDKLCTVAT
LRETYGEMADCCAKQEPERNECFLQHKDDNPNLPRLVRPEVDVMCT
AFHDNEETFLKKYLYEIARRHPYFYAPELLFFAKRYKAAFTECCQAA
DKAACLLPKLDELRDEGKASSAKQRLKCASLQKFGERAFKAWAVA
RLSQRFPKAEFAEVSKLVTDLTKVHTECCHGDLLECADDRADLAKYI
CENQDSISSKLKECCEKPLLEKSHCIAEVENDEMPADLPSLAADFVES
KDVCKNYAEAKDVFLGMFLYEYARRHPDYSVVLLLRLAKTYETTLE
KCCAAADPHECYAKVFDEFKPLVEEPQNLIKQNCELFEQLGEYKFQN
ALLVRYTKKVPQVSTPTLVEVSRNLGKVGSKCCKHPEAKRMPCAED
YLSVVLNQLCVLHEKTPVSDRVTKCCTESLVNRRPCFSALEVDETYV
PKEFNAETFTFHADICTLSEKERQIKKQTALVELVKHKPKATKEQLK
AVMDDFAAFVEKCCKADDKETCFAEEGKKLVAASQAALGL.

TANZEUM™ is an albiglutide formulation for subcutaneous injection. The initial recommended dose for treating type 2 diabetes mellitus is 30 mg SC once weekly, which may be increased to 50 mg once weekly if the glycemic response is inadequate.

e. Dulaglutide

Dulaglutide is GLP-1 receptor agonist that includes a dipeptidyl peptidase-IV-protected GLP-1 analog covalently linked to a human IgG4-Fc heavy chain by a small peptide linker. The amino acid sequence for dulaglutide is:

(SEQ ID NO: 8)
HGEGTFTSDVSSYLEEQAAKEFIAWLVKGGGGGGGSGGGGSGGGGS
AESKYGPPCPPCPAPEAAGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQEE
MTKNQVSLTCLVKGFYPSDIAVEWESNGQPENNYKTTPPVLDSDGSF
FLYSRLTVDKSRWQEGNVFSCSVMHEALHNHYTQKSLSLSLG.

TRULICITY is a dulaglutide formulation for subcutaneous injection. The initial recommended dose for treating type 2 diabetes mellitus is once-weekly SC injection 0.75 mg, which may be increased to 1.5 mg once weekly for additional glycemic control.

f. Semaglutide

Semaglutide is GLP-1 analog that differs to others in the following ways: amino acid substitutions at position 8 (alanine to alpha-aminoisobutyric acid, a synthetic amino acid) and position 34 (lysine to arginine), and acylation of the peptide backbone with a spacer and C-18 fatty di-acid chain to lysine at position 26. These changes permit a high-affinity albumin binding and stabilize semaglutide against dipeptidylpeptidase-4, giving it a long plasma half-life. The amino acid sequence for semaglutide is:

HXEGTFTSDVSSYLEGQAAKEFIAWLVRGRG (SEQ ID NO:9), where X is alpha-aminoisobutyric acid and Lys20 is acylated with C-18 stearic diacid (AEEAc-AEEAc-γ-Glu-17-carboxyheptadecanoyl).

OZEMPIC® is a semaglutide formulation for subcutaneous injection. The initial recommended dose for treating type 2 diabetes mellitus is 0.25 mg SC every week for 4 weeks, then increase the dosage to 0.5 mg weekly.

g. Taspoglutide

Taspoglutide is the 8-(2-methylalanine)-35-(2-methylalanine)-36-L-argininamide derivative of the amino acid sequence 7-36 of human GLP-1. Thus, the sequence of taspoglutide is HXEGTFTSDVSSYLEGQAAKEFIAWLVKXX (SEQ ID NO:10), wherein X2 is 2-methylalanine, X29 is 2-methylalanine, and X30 is L-arginine amide.

Studies show that 20 mg taspoglutide administered once weekly by subcutaneous injection for 4 weeks, followed by dose maintenance at 20 mg, or titration to 30 mg (20/30) or 40 mg (20/40) once weekly for an additional 4 weeks was safe, well tolerated at high doses and efficacious for lowering HbA(1c) (Ratner, et al., Diabet Med. 2010 May; 27(5):556-62. doi: 10.1111/j.1464-5491.2010.02990.x).

(iii). Large Proteins

The bioactive agent can be a large protein. For example, in some embodiments, the protein is at least 100 kDa, at least 110 kDa, at least 120 kDa, at least 130 kDa, at least 140 kDa, at least 150 kDa, etc., up to about 10,000 kDa. However, large molecular weight proteins are generally in the range of about 100 kDa or 150 kDa or 200 kDa up to about 1,500 kDa, or about 1,000 kDa.

In some embodiments, the protein is an antibody. The term antibody is intended to denote an immunoglobulin molecule that possesses a variable region antigen recognition site. The term variable region is intended to distinguish such domain of the immunoglobulin from domains that are broadly shared by antibodies (such as an antibody Fc domain). The variable region includes a hypervariable region whose residues are responsible for antigen binding. The hypervariable region includes amino acid residues from a Complementarity Determining Region or CDR (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). Framework Region or FR residues are those variable domain residues other than the hypervariable region residues as herein defined.

The term antibody includes monoclonal antibodies, multispecific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA, and IgY), class (e.g., IgG₁, IgG₂, IgG₃, IgG₄, IgA₁, and IgA₂) or subclass.

Thus, the term antibody includes both intact molecules as well as fragments thereof that include the antigen-binding site and are capable of binding to the desired epitope. These include Fab and F(ab′)₂ fragments which lack the Fc fragment of an intact antibody, and therefore clear more rapidly from the circulation, and may have less non-specific tissue binding than an intact antibody (Wahl et al., J Nuc. Med. 24:316-325 (1983)). Also included are Fv fragments (Hochman, J. et al., Biochemistry, 12:1130-1135(1973); Sharon, J. et al., Biochemistry, 15:1591-1594 (1976)).

In some embodiments, the disclosed compositions and methods are used to deliver a therapeutic antibody. Therapeutic antibodies include, but are not limited to, those discussed in Reichert, Mabs, 3(1): 76-99 (2011), for example, AIN-457, bapineuzumab, brentuximab vedotin, briakinumab, dalotuzumab, epratuzumab, farletuzumab, girentuximab (WX-G250), naptumomab estafenatox, necitumumab, obinutuzumab, otelixizumab, pagibaximab, pertuzumab, ramucirumab, REGN88, reslizumab, solanezumab, Tih, teplizumab, trastuzumab emtansine, tremelimumab, vedolizumab (ENTYVIO®), zalutumumab and zanolimumab.

Other therapeutic antibodies approved for use, in clinical trials, or in development for clinical use which include, but are not limited to, rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PR070769 (PCT/US2003/040426, entitled “Immunoglobulin Variants and Uses Thereof”), trastuzumab (Herceptin®, Genentech) (see for example U.S. Pat. No. 5,677,171), a humanized anti-Her2/neu antibody approved to treat breast cancer; pertuzumab (rhuMab-2C4, Omnitarge), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); 1CR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1):71-81); mAb-806 (Ludwig Institue for Cancer Research, Memorial Sloan-Kettering) (Jungbluth et al. 2003, Proc Natl Acad Sci USA. 100(2):639-44); KSB-102 (KS Biomedix); MRI-1 (IVAX, National Cancer Institute) (PCT WO 0162931A2); and SC100 (Scancell) (PCT WO 01/88138); alemtuzumab (Campath®, Millenium), a humanized mAb currently approved for treatment of B-cell chronic lymphocytic leukemia; muromonab-CD3 (Orthoclone OKT3@), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amcvive®), anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel@), an p75 TNF receptor Fc fusion developed by Immunex/Amgen, lenercept, an p55TNF receptor Fc fusion previously developed by Roche, ABX-CBL, an anti-CD 147 antibody being developed by Abgenix, ABX-IL8, an anti-IL8 antibody being developed by Abgenix, ABX-MAI, an anti-MUC18 antibody being developed by Abgenix, Pemtumomab (R1549,90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegrene (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-.beta.2 antibody being developed by Cambridge Antibody Technology, ABT 874 (J695), an anti-IL-12 p40 antibody being developed by Abbott, CAT-192, an anti-TGF.beta.1 antibody being developed by Cambridge Antibody Technology and Genzyme, CAT-213, an anti-Eotaxinl antibody being developed by Cambridge Antibody Technology, LyntphoStat-B® an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc. Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech. Xolair® (Omalizurnab), an anti-IgE antibody being developed by Genentech, Raptiva® (Efalizurnab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDFC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-I), and anti-Her2 antibody being developed by Medarex and Immuno-Designed Molecules, HuMaxe-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J. CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAFO, an anti-gamma interferon antibody being developed by Protein Design Labs, Anti-501 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLN01, an anti-Beta2 integrin antibody being developed by Xoma. In another embodiment, the therapeutics include KRN330 (Kirin); huA 33 antibody (A33, Ludwig Institute for Cancer Research); CNTO 95 (alpha V integrins, Centocor); MEDI-522 (alpha V133 integrin, Medimmune); volociximab (aVo1 integrin, Biogen/PDL); Human mAb 216 (B cell glycosolated epitope, NCI); BiTE MT103 (bispecific CD19x CD3, Medimmune); 4G7× H22 (Bispecific BcellxFcgammaRl, Meclarex/Merck KGa); rM28 (Bispecific CD28×MAPG, EP1444268); MDX447 (EMD 82633) (Bispecific CD64×EGFR, Medarex); Catumaxomab (removah) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); THIOMAB (Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCI); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD 122 (CD40, Novartis); SGN-40 (CD40, Seattle Genentics); Campathlh (Alemtuzumab) (CD52, Genzyme); MDX-1411 (CD70, Medarex); hLL1 (EPB-I) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb); Tremelimumab (Ticilimumab, CP-675,2) (CTLA4, Pfizer); 1-IGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF 1-R, Pfizer); IMC-A12 (IGF1-R, Imclone); B1111022 Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTOR, Biogen); HuHMFGI (MUC1, Antisoma/NCI); RAV 12 (N-linked carbohydrate epitope, Raven); CAL (parathyroid hormone-related protein (PTH-rP), University of California); CT-O11 (PD1, CtireTech); MDX-1106 (ono-4538) (PDL Nileclarox/Ono); MAb CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFα, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab-WO/2000/034337, University of Texas); IMC-18F1 (VEGFR1, Imclone); IMC-1121 (VEGFR2, Imclone).

A description of these and other classes of useful agents and a listing of species within each class can be found in Martindale, The Extra Pharmacopoeia, 30th Ed. (The Pharmaceutical Press, London 1993), the disclosure of which is incorporated herein by reference in its entirety.

In some forms, the agent to be delivered can alter the zeta potential of the polymeric particles upon incorporation of the agent into the polymeric particles. Accordingly, and as described above, after loading the agent to be delivered, the particles have a zeta potential between −20 mV and −70 mV, inclusive.

Every polymer, polymeric particle, and formulation referenced herein are intended to be and should be considered to be specifically disclosed herein.

Further, every subgroup that can be identified within the above definition is intended to be and should be considered to be specifically disclosed herein. As a result, it is specifically contemplated that any polymer, polymeric particle, and formulation or subgroup of polymers, polymeric particles, and formulations can be either specifically included for or excluded from use or included in or excluded from a list of polymers, polymeric particles, and formulations. For example, any one or more of the polymers, polymeric particles, and formulations described herein, with a structure depicted herein, or referred to in the Figures the Examples herein can be specifically included, excluded, or combined in any combination, in a set or subgroup of such polymers, polymeric particles, and formulations. Such specific sets, subgroups, inclusions, and exclusions can be applied to any aspect of the compositions and methods described here. For example, a set of polymers, polymeric particles, and formulations that specifically excludes one or more particular polymers, polymeric particles, and formulations can be used or applied in the context of polymers and polymeric particles per se (for example, a list or set of polymers and polymeric particles), formulations including the polymers or polymeric particles, any one or more of the disclosed methods, or combinations of these. All of these different sets and subgroups of polymers, polymeric particles, and formulations are specifically and individual contemplated and should be considered as specifically and individually described. For example, the following can be specifically included or excluded, as a group or individually, from any polymers per se (for example, a list or set of polymers), polymeric particle or formulations or any one or more of the disclosed methods, or combinations of these. For example the polymers and polymeric particles can exclude a polymer that readily dissolves in water, such as poly(butadiene-maleic anhydride-co-L-dopamine) or poly(ethylene glycol); copolymers of the polymers and polymeric particles containing these copolymers can exclude poly(ethylene glycol); and further, polymeric particles can exclude polyethylene glycol covalently bound on their surface.

### II. Methods of Making Polymeric Particles and Reagents Therefor

The polymeric particles can be manufactured using any method in the art, such as single step double-walled nanoencapsulation (SSDN). SSDN is described in detail in Azagury, et al., Journal of Controlled Release 280, (2018), 11-19, the contents of which are incorporated herein by reference. Preferably, the polymeric particles are manufactured via phase inversion nanoencapsulation (PIN) phenomenon. U.S. Patent Application Publication US20040070093 A1 by Mathiowitz, et al., describe phase inversion, the contents of which are hereby incorporated by reference.

Briefly, phase inversion is a physical process in which a polymer is first dissolved in “good” solvent, forming one continuous homogenous liquid phase. By adding this mixture to the excess of a non-solvent (or “bad” solvent), an unstable two-phase mixture of polymer rich and polymer poor fractions is formed, causing the polymer to aggregate at the nucleation points. When the polymer concentration reaches a certain point (cloud point), polymeric particles phase separate, solidifying and precipitating from the solution.

Unlike solvent removal or solvent evaporation methods, PIN does not require emulsification of the initial continuous phase polymer/solvent solution. It utilizes low polymer concentrations and low viscosities of the encapsulants. Also, the solvent and non-solvent pairs are preferably miscible with at least ten times excess of non-solvent relative to solvent. These conditions allow for rapid addition of polymer dissolved in continuous solvent phase into non-solvent, which in turn result in spontaneous formation of nanomaterial or micromaterial. Since no emulsification is required in this process and the nanospheres or microspheres form spontaneously, the size of the resulting spheres is controlled not by the speed of stirring, but rather by changing the parameters of the procedure: polymer concentration, solvent to non-solvent ratio and their miscibility.

Any method of encapsulation known to those of skill in the art could be used for the non-biologics agents, e.g. solvent evaporation (e.g. emulsion and solvent evaporation), nanoprecipitation, microfluidics, self-assembly, solvent diffusion/displacement, solvent removal, spray drying, etc. Preferably, for biologics (e.g. proteins), PIN can be used since the activities of the biologics are retained.

### IV. Methods of Using

The polymeric particles can be administered in formulations, or used to prepare formulations, for the treatment of diseases or disorders that may or may not be associated with the GI tract. The formulations can be prepared using a pharmaceutically acceptable carrier composed of materials that are considered safe and effective and may be administered to an individual without causing undesirable biological side effects or unwanted interactions. The carrier is all components present in the pharmaceutical formulation other than the agent to be delivered and the polymeric particles. The term “carrier” includes but is not limited to diluents, binders, lubricants, disintegrators, and fillers.

Suitable pharmaceutically acceptable carriers include any of the standard pharmaceutical carriers, such as a phosphate buffered saline solution, water, and emulsions such as an oil/water or water/oil emulsion, and various types of wetting agents. The formulations containing the polymeric particles can also be in solid dosage forms.

Preferred methods of administration of the formulations are oral, i.e., administration to or by way of the mouth, to provide uptake through the GI tract; or enteral, i.e., administration directly to the intestines. In some forms, such as in experimental setting, enteral administration can be by injection to the intestines. In systemic circulation, the polymeric particles may preferentially accumulate in diseased site.

Typically, the polymeric particles contain an effective amount of an agent to be delivered for treating and/or preventing a given disease or disorder. The formulations can be administered in a single dose or in multiple doses. Certain factors may influence the dosage required to effectively treat or prevent a disease or disorder, including, but not limited to, the severity of the disease or disorder, previous preventions, the general health and/or age of the subject, and other diseases present. It will also be appreciated that the effective dosage of the composition used for prevention may increase or decrease over the course of particular prevention. Changes in dosage may result and become apparent from the results of assays.

Preventing or prevention includes administering the polymeric particles or a formulation containing the particles to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, or stabilization or delay of the development or progression of the disease or disorder, or to have a combination of these effects.

i. Diseases or Disorders to be Treated

The formulations described herein can be administered to a subject to treat any disease or disorder or ameliorate one or more symptoms associated with a disease or disorder.

The subject or patient is an individual who is the target of treatment using the disclosed formulations or polymeric particles. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can also include a control subject or a test subject.

Diseases or disorders that can be treated include, but are not limited to, diabetes; autoimmune disorders (e.g. Crohn's disease, chronic arthritis, multiple sclerosis, Sjogren's disease, Lupus erythematosus, psoriasis, Celiac disease, etc); cancer (breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's or non-Hodgkin's lymphoma), leukemia (e.g., acute myeloid leukemia) and gastrointestinal cancer); pain; fungal infections; bacterial infections; inflammation; anxiety; etc.

The disclosed polymeric particles, formulations, and methods can be further understood through the following numbered paragraphs.
1. Polymeric particles comprising an active agent encapsulated therein,
wherein the polymeric particles have a zeta potential between −10 mV and −80 mV, between −15 mV and −70 mV, between −20 mV and −70 mV, between −20 mV and −60 mV, between −30 mV and −60 mV, or between −40 mV and −60 mV, and a diameter between 100 nm and 5000 nm, inclusive, between 100 nm and 2000 nm, inclusive, and wherein the zeta potential is measured in DI water at room temperature and pH of between about 5 and about 7.4, or between 5 and 6, inclusive, using a Zetasizer/Zetaview.
2. The polymeric particles of paragraph 1, comprising a moiety that imparts a negative zeta potential to the polymeric particles, wherein the moiety is bonded to (i) a polymer, or (ii) the active agent encapsulated therein.
3. The polymeric particles of paragraph 1 or 2, further comprising a polymer, wherein the polymer (i) is incorporated in a polymeric matrix that forms the polymeric particles or (ii) is coated on the surface of the polymeric particles.
4. The polymeric particles of paragraph 3, wherein the polymer is hydrophobic.
5. The polymeric particles of paragraph 3 or 4, wherein the polymer does not dissolve in water within one hour at a pH between 6 and 7, inclusive, at room temperature, preferably wherein the polymer is non-soluble in a medium having a pH between 1 and 7, inclusive, five minutes, 10 minutes, 15 minutes, 20 minutes, 30 minutes, 45 minutes, or one hour after the polymer contacts the medium.
6. The polymeric particles of any one of paragraphs 3 to 5, wherein the polymer is biodegradable and biocompatible.
7. The polymeric particles of any one of paragraphs 3 to 6, wherein the polymer has a molecular weight between 1.5 kDa and 300 kDa, inclusive, 1.5 kDa and 275 kDa, inclusive, 1.5 kDa and 250 kDa, inclusive, between 1.5 kDa and 100 kDa, between 2 kDa and 80 kDa, inclusive, between 2 kDa and 50 kDa, inclusive, between 2 kDa and 30 kDa, inclusive, between 2 kDa and 20 kDa, or between 2 kDa and 10 kDa, inclusive, preferably about 2 kDa, about 2.5 kDa, about 5 kDa, or about 8 kDa.
8. The polymeric particles of any one of paragraphs 3 to 7, wherein the polymer comprises a blend of a low molecular weight polymer having a molecular weight between 2 kDa and 20 kDa, between 2 kDa and 15 kDa, or between 2 kDa and 10 kDa, and a high molecular weight polymer having a molecular weight between 21 kDa and 300 kDa.
9. The polymer of paragraph 8, having a ratio of the low molecular polymer to the high molecular polymer between 30% wt/wt and 90% wt/wt, inclusive, between 40% wt/wt and 90% wt/wt, inclusive, between 50% wt/wt and 90% wt/wt, inclusive, between 60% wt/wt and 90% wt/wt, inclusive, between 70% wt/wt and 90% wt/wt, inclusive, or between 80% wt/wt and 90% wt/wt, inclusive.
10. The polymeric particles of any one of paragraphs 3 to 9, wherein the polymer is selected from the group consisting of polyesters, such as poly(caprolactone); poly(hydroxyacids), such as poly(lactic acid), poly(glycolic acid), and poly(lactic acid-co-glycolic acid); polyhydroxyalkanoates, such as poly(3-hydroxybutyrate) and poly(4-hydroxybutyrate); polyanhydrides (poly(fumaric-co-sebacic acid), polysebacic acid, polyfumaric acid); poly(orthoesters); hydrophobic polypeptides; hydrophobic polyethers, such as poly(propylene oxide); poly(phosphazenes), polyesteramides, poly(alkylene alkylates), polyether esters, polyacetals, polycyanoacrylates, polyketals, polyhydroxyvalerates, polyalkylene oxalates, polyalkylene succinates, mixtures, and copolymers thereof.
11. The polymeric particles of any one of paragraphs 3 to 10, wherein the polymer is selected from the group consisting of poly(lactic acid), poly(fumaric-co-sebacic acid), poly(glycolic acid), poly(lactic acid-co-glycolic acid), polysebacic acid, polyfumaric acid, mixtures, and copolymers thereof.
12. The polymeric particles of any one of paragraphs 2 to 11, wherein the moiety that imparts a negative charge is an acidic or anionic group, peptides, amino acids, lipids, salts, or combinations thereof.
13. The polymeric particles of any one of paragraphs 2 to 12, wherein the moiety that imparts a negative charge is selected from the group consisting of carboxylic acids, protonated sulfates, protonated sulfonates, protonated phosphates, singly- or doubly protonated phosphonates, and singly- or doubly protonated hydroxamate, carboxylates, sulfates, sulfonates, singly- or doubly deprotonated phosphate, singly- or doubly deprotonated phosphonate, and hydroxamate.
14. The polymeric particles of any one of paragraphs 2 to 13, wherein the moiety that imparts a negative charge is covalently attached to the polymer.
15. The polymeric particles of any one of paragraphs 2 to 14, wherein the polymer is bioadhesive, preferably wherein the polymer has a bioadhesion force of about 500 mN/cm² (such as 480 mN/cm²), or greater.
16. The polymeric particles of any one of paragraphs 1 to 15, wherein the size of the polymeric particles is between 100 nm and 800 nm, between 100 nm and 500 nm, between 200 nm and 400 nm, between 900 nm and 2000 nm, between about 1000 nm and 2000 nm, between 1200 nm and 2000 nm, between 1300 nm and 1800 nm.
17. The polymeric particles of any one of paragraphs 1 to 15, further comprising one or more anionic surfactants, peptides, lipids, amino acids, salts, or combinations thereof.
18. The polymeric particles of paragraph 17, wherein the anionic surfactants, peptides, lipids, amino acids, salts, or combinations thereof, constitute between about 0.0001% wt/wt and about 5% wt/wt, between about 0.001% wt/wt and about 5% wt/wt of the polymeric particles.
19. The polymeric particles of paragraph 17 or 18, wherein the anionic surfactant is petroleum sulfonate, naphthalenesulfonate, olefin sulfonate, an alkyl sulfate, sulfated natural oil, sulfated fat, sulfated ester, a sulfated alkanolamide, a sulfated alkylphenol, a sulfated alkylphenol ethoxylate, laureate, lauryl ether sulfate, lauryl sulfate, decyl sulfate, octyl sulfate, a alkylbenzene sulfonate (a linear alkylbenzene sulfonate, or a branched alkylbenzene sulfonate, or a combination thereof), or a combination thereof.
20. The polymeric particles of any one of paragraphs 1 to 19, wherein the active agent is selected from the group consisting of small molecules, proteins, polypeptides, peptides, carbohydrates, nucleic acids, glycoproteins, lipids, antibodies/antigens, and combinations thereof.
21. The polymeric particles of any one of paragraphs 1 to 20, wherein the polymeric particles show systemic uptake between 10% and 80%, between 10% and 70%, between 20% and 75%, between 20% and 70%, between 30% and 70%, or between 30% and 60% in a mammal, as measured using Fourier Transform Infrared spectroscopy.
22. The polymeric particles of any one of paragraphs 1 to 21, wherein the active agent is not glucagon-like peptide-1 (GLP-1) or a truncated biologically active portion thereof or an analog thereof.
23. The polymeric particles of any one of paragraphs 1 to 21, wherein the active agent is glucagon-like peptide-1 (GLP-1) or a truncated biologically active portion thereof or an analog thereof, and wherein the polymeric particle does not contain PAA (poly-adipic acid), PLGA (poly-lactic-co-glycolic acid), or PLA (poly-lactic acid) as the sole polymer forming the polymeric particle.
24. A formulation comprising the polymeric particles of any one of paragraphs 1 to 23, and a pharmaceutically acceptable carrier.
25. The formulation of paragraph 24, wherein the size of the polymeric particles is between 100 nm and 800 nm, between 100 nm and 500 nm, between 200 nm and 400 nm, between 900 nm and 2000 nm, between about 1000 nm and 2000 nm, between 1200 nm and 2000 nm, between 1300 nm and 1800 nm.
26. The formulation of paragraph 24, further comprising anionic surfactants, peptides, lipids, amino acids, salts, or combinations thereof.
27. The formulation of paragraph 26, wherein the anionic surfactants, peptides, lipids, amino acids, salts, or combinations thereof, constitute between about 0.0001% wt/wt and about 5% wt/wt, between about 0.001% wt/wt and about 5% wt/wt of the polymeric particles.
28. The formulation of paragraph 26, wherein the anionic surfactant is petroleum sulfonate, naphthalenesulfonate, olefin sulfonate, an alkyl sulfate, sulfated natural oil, sulfated fat, sulfated ester, a sulfated alkanolamide, a sulfated alkylphenol, a sulfated alkylphenol ethoxylate, laureate, lauryl ether sulfate, lauryl sulfate, decyl sulfate, octyl sulfate, a alkylbenzene sulfonate (a linear alkylbenzene sulfonate, or a branched alkylbenzene sulfonate, or a combination thereof), or a combination thereof.
29. The formulation of paragraph 24, wherein the active agent is selected from the group consisting of small molecules, proteins, polypeptides, peptides, carbohydrates, nucleic acids, glycoproteins, lipids, antibodies/antigens, and combinations thereof.
30. The formulation of paragraph 24, wherein the polymeric particles show systemic uptake between 10% and 80%, between 10% and 70%, between 20% and 75%, between 20% and 70%, between 30% and 70%, or between 30% and 60% in a mammal, as measured using Fourier Transform Infrared spectroscopy.
31. The formulation of paragraph 24, wherein the active agent is not glucagon-like peptide-1 (GLP-1) or a truncated biologically active portion thereof or an analog thereof.
32. The formulation of paragraph 24, wherein the active agent is glucagon-like peptide-1 (GLP-1) or a truncated biologically active portion thereof or an analog thereof, and wherein the polymeric particle does not contain PAA (poly-adipic acid), PLGA (poly-lactic-co-glycolic acid), or PLA (poly-lactic acid) as the sole polymer forming the polymeric particle.
33. The polymeric particles of any one of paragraphs 1 to 23 or the formulation of any one of paragraphs 24 to 32 for use in the method of treating a human or animal body by therapy, wherein the method is for delivering an active agent, and comprises administering the polymeric particles or the formulation to the patient.
34. A method of administering therapeutic agents, prophylactic agents, or diagnostic agents to a subject in need thereof, comprising administering to the subject, the polymeric particles of any one of paragraphs 1 to 23 or the formulation of paragraph 24.
35. The method of paragraph 34, wherein the polymeric particles are administered orally.

Those skilled in the art will recognize, or be able to ascertain using no more than routine experimentation, many equivalents to the specific embodiments of the invention described herein. Such equivalents are intended to be encompassed by the ensuing claims.

The present invention will be further understood by reference to the following non-limiting examples.

### EXAMPLES

### Example 1. The Effects of Environment (Water and Mucin) on the Zeta Potentials (Surface Charge) of the Polymeric Particles

### Materials and Methods

Materials were purchased from Fisher-scientific or Polysciences. Measurements were all made using a Malvern Zetasizer. Blank polymeric nanospheres were prepared using Phase Inversion Nanoencapsulation method (PIN), developed and patented by Mathiowitz lab (Mathiowitz, Chickering, et al.). Around 80 mg of bulk polymeric material was dissolved in 5.3 mL of dichloromethane (DCM), keeping the ratio of polymer to DCM at 1.5% w/v. DCM, served as “good” solvent for all the polymers used, apart from PBMAD (ethanol was used instead). The solution was vortexed for about 30 seconds and then sonicated for 30 seconds using Ultrasonic Homogenizer CV26 (Cole-Palmer; Vernon-Hills, IL) until the polymer was completely dissolved, resulting in a clear solution. Depending on the polymer used, more vortexing/sonication rounds might have been performed for complete dissolution of the material. The obtained solution was then introduced dropwise or continuously to an excess of “bad” solvent (or non-solvent), which in this case is 660 mL of petroleum ether (PE). In all productions, the volumetric ratio of solvent to non-solvent was kept at 1:100. The solution was stirred using magnet stirrer with enough speed to create a vortex in the tall 1000 mL glass beaker for around 5-7 minutes or until the material started to aggregate into large snowflakes. The entire solution was then run through the positive pressure filtration column with 0.2 μm PTFE filter (Millipore; Billerica, MA) to collect the resulting nanospheres. They were then scraped from the filter, flash frozen and lyophilized for at least 24 hours to remove the residual solvents. The PTFE filters were subjected to flash freezing and lyophilization as well to be used in SEM sample preparation. All PIN products were afterward stored at −20 °C.

### Results

All the particles have a negative charge in water, ranging from −17 mV to −53 mV (FIG. 1). In mucin, however, the charge is significantly reduced. While the charge of mucin without any nanomaterial was found to be −7.3 mV, the charge of nanospheres in mucin ranges from −16 to −7.7 mV. PMMA and PEG-PLGA stand out by having much lower effective charge in water, compared to the rest for the tested polymers. As a result, there was no substantial charge reduction in mucin compared to water for these two polymers.

Based on these data, mucin has a masking effect on the particle zeta potential measurement. Hence, it appears that mucin has the ability to coat the particles and, thus, skew the surface charge measurement towards a less negative value (i.e., values similar to the control measurement of mucin alone, FIG. 1).

### Example 2. The Relationship Between Charge and Bioadhesion Force or Bioadhesion Work

### Materials and Methods

Bioadhesion measurements were performed on the polymeric particles manufactured in Example 1. FIGS. 2A-2D contain bioadhesion data that were obtained via in vitro experiments with rat tissue. Tensile testing was done using the Texture Analyzer TA.XTplus (Stable MicroSystems, Godalming, UK) and corresponding Texture Exponent software. Standard straight pins with spherical glass heads and nickel-plated steel pin bodies were used as the probes. Intestinal tissue was excised from adult male Sprague-Dawley rats immediately post-mortem.

Polymer Solution Preparation:

Each polymer solution of 5% weight/volume was prepared in a glass scintillation vial by adding 300 mg of polymer to 5 mL of solvent. Solvents were chosen based on known solubilities and introduced to the glass heads of the pin probes. Dichloromethane (DCM) was chosen as the solvent for polystyrene, FASA 20:80, polyaspirin, PLGA, PLA, and PMMA. Both ethanol and acetone could be used as the solvent for PBMAD. In this example, PBMAD was dissolved in acetone.

Pin Coating:

To coat the glass pinheads, which would come in contact with intestinal tissue, with the desired polymer, each pin was individually dipped into the polymer solution. After being fully submerged in the solution, the pin was immediately removed and placed upright on a Styrofoam block in a fume hood to allow for even drying. A minimum of 30 minutes was allotted for drying in between dippings. Each pin was dipped into the solution a minimum of five times. After all dippings and drying was complete, calipers were used to measure the diameter of all pins.

### Tissue Removal and Preparation:

An Albino, Sprague-Dawley male rat was fasted for 24 h prior to surgery. Rats were euthanized using CO₂ asphyxiation and subsequent excision of the diaphragm. The anterior abdomen was shaved and an incision was made along the sternum. Then, the small intestinal tissue was harvested as follows. The first cut was made at the junction of the ileum and cecum. The second cut was made at the pyloric sphincter. The length of the small intestine is then removed, cutting mesenteric tissue as necessary, and placed in a petri dish containing phosphate-buffered saline (PBS). The PBS provided a neutral environment for the intestines and prevented the tissue from sticking to itself and drying out. The intestine was divided into three sections (at a ratio of 1:2:2 correlating to duodenum:jejunum:ileum), assuming that the middle of the first third (closest to the pyloric sphincter) was duodenum, the middle of the second third was jejunum, and the middle of the final third (closest to the cecum) was ileum. A P1000 pipet was used to push 1 mL PBS through each section. Gently, the feces were squeezed out from each section, with PBS washes repeated until all fecal residue was removed. The cleared intestinal sections were each divided into segments of approximately 3 cm each. Each 3 cm segment then was cut along the anti-mesenteric border so as to expose the lumen. Segments were placed in PBS and on ice until needed.

### Tissue Mounting

To keep the tissue from sticking to the stage, 1 mL of PBS was added to the tissue chamber portion of the stage. The 3 cm tissue segment was placed into the tissue chamber with the lumen (mucus) facing upward. The tissue was secured via metal clamps. An additional 2 mL of PBS at a temperature of 37 °C was added to the chamber to fully submerge the tissue.

### Bioadhesion Testing

The Texture Analyzer TA.XTplus was properly calibrated using known weights. The machine was fitted with a probe that contained a vise with which pins could be mounted. The program was set to move the probe downwards (towards the tissue) at 5 mm per second. The contact force was set to 5 g. Once the contact force was reached, the probe would stop moving and remain in contact with the tissue for a predetermined amount of time. The contact time used was 420 seconds (7 minutes), as was previously used in the lab (Chickering and Mathiowitz, Journal of Controlled Release (1995), 34: 251-261; Laulicht, et al., Macromolecular bioscience (2012), 12(11), 1555-1565). This amount of time was chosen to allow for any polymer hydration or bond formation that might occur, though some polymers may exhibit optimal bioadhesion after more time (Estrellas, et al., Colloids and Surfaces B: Biointerfaces (2019), 173, 454-469). Once the contact time had elapsed, the probe was moved upwards (away from the tissue) at 5 mm per second while recording the forces between the probe and tissue. After each test, the used pin was disposed of. More than one test (1-3) can be performed for each 3 cm segment of tissue, so long as a fresh area is used for each test.

### Results

The results are incorporated and shown in FIGS. 2A-2D. The data presented in these figures show no direct correlation between the negative zeta potential of the polymeric particles and their respective bioadhesion work nor bioadhesion force. Both PS and PLA particles which are not typically considered to be strong bioadhesive materials (displaying low bioadhesion force and bioadhesion work values) showed zeta potential charges on the same order as the known bioadhesive materials PBMAD and PFASA (which displayed high bioadhesion force and bioadhesion work values). Thus, while the embodied invention includes bioadhesivity, this is not the predominant property. The negative zeta potential predominates. In other words, and without wishing to be bound by any particular theory, it may be that all good bioadhesive polymers will have the desired negative charge but not all negatively charged polymeric particles are good bioadhesive materials.

### Example 3. Polymeric Particle Size and Absorption in the GI

### Materials and Methods

For in vitro experiments, an Albino, Sprague-Dawley male rat was fasted for 24 h prior to surgery. Rats were euthanized using CO₂ asphyxiation and subsequent excision of the diaphragm. The anterior abdomen was shaved and an incision was made along the sternum followed by harvesting of the small intestine. Then, the intestine was divided into the duodenum, jejunum, and ileum sections (~1:2:2 ratios respectively). To ensure proper removal of fecal matter, the sections were rinsed each with approximately 3 mL of PBS. Then each section was bisected, splayed and cut into several sections of 2 cm each. For this section procedure, the duodenum was cut into 4 cm long samples. Each of these pieces was cut into 2 cm long samples (with and without mucus). Next, the splayed tissue sections were pinned to PDMS (wetted with PBS) blocks for exposure. Then, each of the tested polymeric particles was dispersed in 200 μL of phosphate-buffered saline (PBS, 0.01 M) at a concentration of 75 mg/mL (15 mg total). Mixtures were sonicated and vortexed to facilitate the dispersion within the buffer prior to use. The tissue samples were exposed to polymeric particle dispersions for 1 h. To investigate the effect of the loose mucus on polymeric particle absorption, half of the sections had their loose mucus removed prior to exposure using a scalpel gently not to harm the tissue. Post-exposure, all samples were washed gently with approximately 3 mL of sterile PBS to flush unabsorbed polymer, then flash frozen and lyophilized to remove moisture content for FTIR analysis. In addition, control unexposed tissue samples were collected for each of the sections. Another option could be using the dried lyophilized tissue via attenuated total reflectance (ATR) FTIR. Other methods of detection and quantification could include but not limited to: LC-MS, GPC, HPLC, MALDI-TOF, or via labeling the particles (e.g. radiolabeled, fluorescent labeled)

The polymer concentration in mucus was determined by diluting the mucus, then centrifuging it at 10000 g for 8 min. The supernatant was then discarded and the pellet was dried and weighted (sample was also analyzed by FTIR to confirm it is the tested polymer). Another option could be using the dried lyophilized tissue via attenuated total reflectance (ATR) FTIR. Other methods of detection and quantification could include but not limited to: LC-MS, GPC, HPLC, MALDI-TOF, or via labeling the particles (e.g. radiolabeled, fluorescent labeled).

### In Vivo Detection and Quantification of Polymeric Particles in GI Tract of Rats

First, Sprague-Dawley rat was fasted for 24 h prior to surgery. The rat was anesthetized and the anterior abdomen was shaved and opened through an incision along the sternum. With the rat under anesthesia, a region approximately 40 cm upstream from the cecum was selected for isolated loop. A knot was tightly placed in the chosen segment, and a second knot was loosely placed approximately 10 cm upstream. Polymeric particles suspended in PBS solution were injected into the isolated loop and the upper knot was tightened to seal the section. Isolated loops were left to absorb for 5 h. Once completed, the isolated loops were removed from the GI tract. Then, the knots were removed and the inner tracts were rinsed with approximately 3 mL of PBS. In addition, for every isolated loop, another section (near to isolated loop) was harvested to serve as control tissue. After rinsing, each section was opened through axial incision. The loose mucus from each loop was removed and collected by gently scraping it. The loose mucus, exposed tissue, and control tissue were collected in pre-weighed vials, flash frozen, and lyophilized. After lyophilization, each vial weight was collected to calculate the tissue dry weight. Another option for detection and quantification could be using the dried lyophilized tissue via attenuated total reflectance (ATR) FTIR. Other methods of detection and quantification could include but not limited to: LC-MS, GPC, HPLC, MALDI-TOF, or via labeling the particles (e.g. radiolabeled, fluorescent labeled).

### Results

The results are shown in FIGS. 3A-3C. FIG. 3A presents the absorption of PS particles in the different regions (duodenum: jejunum: ileum) of the rat's GI post ex vivo exposure. It shows that the duodenum showed the highest penetration of PS particles compared to the other regions (2.65% vs 0.34% and 0%) while the mucus that trapped the most of PS was in the ileum (10% vs 8.8 and 1%). This approach could be utilized to other polymeric particles in order to optimize its absorption profile. It also serves as another proof for the importance of mucus in polymeric particles absorption. FIG. 3B, presents the effect of size on the absorption of polymeric particles specifically PS specifically in the ileum section. Two sizes were tested, ‘big’ particles with 1541±151 nm diameter and the ‘small’ with 310±100 nm diameter. Interestingly more of the bigger particles seemed to penetrate the ileum both to the tissue and trapped in the mucus. However, these results are ex vivo meaning no active transport occurred. In addition, these are mass calculations, but in terms of number of particles the smaller still penetrate more (5 times smaller correlates to 125 times more particles).

### Example 4. Polymeric Particle Detection in Blood

Fourier transform infrared (FTIR) spectroscopy can be used for quantitative analysis of a polymeric composition and concentration. The use of FTIR provides the benefits of rapid and low-cost results, and is often more accessible than more complex spectroscopy methods which utilize chromatography. Based on the Lambert-Beer law, the intensity of an FTIR absorption band is directly proportional to the concentration of the component which provides the band. Thus, the concentration of an analyte within a sample can be determined through a calibration curve constructed from analytical standards.

Some polymeric particles may not be readily detected in blood using FTIR if the corresponding absorbance spectrum does not contain an identifiable peak when mixed with blood. Thus, in instances where FTIR cannot be used to detect the polymeric particles in blood, other validated methods including but not limited to GPC, HPLC, mass spectrometry (MS), LC-MS, or via labeling the particles (e.g. radiolabeled, fluorescent labeled) can be employed.

The ability to quantify a polymer analyte in blood is demonstrated in this example through the validation of an FTIR spectroscopy method based on the Lambert-Beer law.

Typical analytical characteristics used in method validation include:

### Specificity

The ability to specifically measure the polymer analyte of interest in the presence of blood serum components must be demonstrated. This can be achieved by qualitatively assessing the FTIR spectra of each raw material and identifying the polymer absorption peak band which has minimal interference by the blood serum. Spiked samples of polymer in blood are then necessary to demonstrate that the polymer peak persists in the presence of blood serum.

### Robustness

The robustness of the method describes the capacity of the results to remain unaffected by small deliberate variations in method parameters (i.e. a measure of the reliability of the method). Variations in blood samples from different sources, sample weights, and sample preparation must be addressed to determine if there is a significant effect on the results of the analysis.

### Linearity and Range

The test results from the method must be proportional to the polymer concentration within a given range. The range of quantification is defined as the interval between the upper and lower levels of polymer concentration that can be determined with suitable levels of precision, accuracy, and linearity. This can be determined by analyzing analytical standards of polymer spiked into blood at a range of concentrations, allowing for linear regression to be fit.

### Accuracy

The accuracy of the method describes how close the test results are to the true value. The percent recovery of the known added amount of polymer in blood should be determined based on the best fit linear regression.

### Precision

The precision of the method describes the degree of agreement among test results when the analytical method is repeated on multiple homogenous samples (i.e. the repeatability of the method). Variations based on random events such as different days, analysts, and equipment should also be assessed. The overall precision of the method is determined by the statistical significances of the standard deviation and relative standard deviation. A common method for ensuring precision within FTIR analysis is the Internal Standard Method, in which the ratio of the polymer analyte peak to the blood serum background peak is plotted rather than the absolute polymer analyte peak alone. This method helps correct for small variations in sample thickness and composition.

### Detection Limit

The limit of detection is the lowest concentration of an analyte in a sample that could be detected (however, not quantified). The detection limit can be determined by analyzing known concentrations of polymer spiked into blood and establishing the minimum level at which the polymer can be reliably detected. The signal to noise ratio of the polymer peak against the surrounding blood peaks should be assessed, as well as the relative standard deviation. An appropriate number of samples should be analyzed at the limit of detection to validate this level.

### Quantification Limit

The limit of quantification is the lowest concentration of an analyte in a sample that can be determined with acceptable accuracy and precision. The value is often determined to have a significantly higher signal to noise ratio than the limit of detection. In this case, the FTIR detection of materials in blood quantification limit was determined based on the calibration curves lowest concentration that could be used in the subtraction method (see details below).

Three peak ratios were chosen. At 1750, 1084, and 1188 cm⁻¹ all are found in the pure PLA FTIR and compared to 1650 cm⁻¹ peak which is a typical peak of the dry serum. Peaks were recorded after baseline correction and normalization were applied on the spectrographs. Since the highest is of the dry plasma at 1650 cm⁻¹, the spectrographs were normalized to this peak giving it the value of one. Hence, all other peak heights at the aforementioned peaks for PLA are actually the peak ratios as well (this is equivalent to taking ratios between the PLA absorption height and the Tissue absorption height at 1650 cm⁻¹. Once peak absorptions were obtained, the control peak ratios were subtracted from or divided by the standards peak ratios (see Tables 1 & 2).

TABLE 1
Subtracted calibration curves numbers for three chosen peaks at
1750, 1084, and 1188 cm⁻¹. Numbers represent at least three repetitions.

| Percent | 1750 | 1084 | 1188 | 1750 Sub | 1084 Sub | 1188 Sub |
|---|---|---|---|---|---|---|
| 1.0 | 0.0409 | 0.1679 | 0.1037 | 0.00996 | 0.0122 | 0.0159 |
| 2.5 | 0.0525 | 0.1583 | 0.1033 | 0.02152 | 0.0026 | 0.0155 |
| 5.0 | 0.0847 | 0.1894 | 0.1263 | 0.05372 | 0.0337 | 0.0385 |
| 7.5 | 0.1216 | 0.2123 | 0.1394 | 0.09061 | 0.0566 | 0.0516 |
| 10.0 | 0.1516 | 0.2520 | 0.1667 | 0.12070 | 0.0963 | 0.0789 |
| 12.5 | 0.2209 | 0.3025 | 0.2036 | 0.18991 | 0.1468 | 0.1158 |
| 15.0 | 0.2940 | 0.3344 | 0.2256 | 0.26309 | 0.1787 | 0.1379 |

TABLE 2
Divided calibration curves numbers for three chosen peaks at 1750,
1084, and 1188 cm⁻¹. Numbers represent at least three repetitions. A
red font represents the limit of detection for that specific wavelength.

| Percent | 1750 | 1084 | 1188 | 1750 Div | 1084 Div | 1188 Div |
|---|---|---|---|---|---|---|
| 1.0 | 0.0409 | 0.1679 | 0.104 | 1.3219 | 1.0783 | 1.1813 |
| 2.5 | 0.0525 | 0.1583 | 0.1033 | 1.6955 | 1.0168 | 1.1766 |
| 5.0 | 0.0847 | 0.1894 | 0.1263 | 2.7362 | 1.2166 | 1.4391 |
| 7.5 | 0.1216 | 0.2123 | 0.1394 | 3.9281 | 1.3632 | 1.5883 |
| 10.0 | 0.1516 | 0.2520 | 0.1667 | 4.9006 | 1.6186 | 1.8993 |
| 12.5 | 0.2209 | 0.3025 | 0.2036 | 7.1373 | 1.9426 | 2.3192 |
| 15.0 | 0.2940 | 0.3344 | 0.2256 | 9.5022 | 2.1478 | 2.5703 |

From the data in Tables 1& 2, six calibration curves were obtained (presented in FIGS. 4B-4G). The peaks' height was taken within ±5 cm⁻¹. As can be seen, the 1750 cm⁻¹ peak seems to have the best calibration based on its R² value.

### Materials and Methods

Isolated loop experiments were performed as described previously (under In Vivo Detection and Quantification of Polymeric Particles in GI Tract of Rats) and as described in Reineke, et al., Proceedings of the National Academy of Sciences of the United States of America 110.34 (2013): 13803-13808, Supplemental Information. Briefly, rats were anesthetized. At zero time point, blood was extracted from the rats. Subsequently, the abdominal cavity of each rat was surgically opened to expose intestines, and 80-120 mg of PLA particles in 1 mL of PBS/DI water were injected into an isolated loop of 10 cm. After five hours blood samples were collected from the rats for detection in blood samples. Subsequently, the rats were euthanized and the isolated loop (ileum or jejunum) was harvested for GI detection. In these studies, n=5. Serum was separated by allowing the blood to clot for 20-25 minutes followed by centrifugation at 4 °C for 18 min and 1500-2000 g. Serum sample was taken carefully from the top (the serum is the resulting supernatant), flash froze and lyophilized. Another option to sample prep biofluids (i.e., blood) is by placing 50-250 μL of blood sample on frosted slides, allowed to be fully dried and then analyzed via ATR-FTIR.

### Results

As a non-limiting example using PLA, this section was focused on calibration of polymeric PLA particles in blood (serum and red blood cells). Other polymers such as P(FA:SA) 20:80, and a monomer such as fumaric monomer were detected as well. Fumaric acid monomer was tested to determine whether the results would be different from what would be observed with poly(fumaric-co-sebacic acid) polymer.

From the calibration curves created for the PLA particles (FIGS. 4B-4G), serum was chosen as a model to test against in vivo experiments (more specifically isolated loop experiments). Serum detection was used as nothing was detected in the red blood cells pellet. The main difficulty in creating a serum calibration curve, is that it tends to stick to both the mortar during the grinding process (in the case of working with dried blood, which is not the case when preping for ATR-FTIR) and the dye during the pressing process. This difficulty was overcome using the liquid method for the creation of the serum calibration curve. Here, each of the three components (KBr, PLA, Blood serum) were suspended and dissolved in water, and mixed in appropriate amounts and ratios (PLA: serum) to create individual points on the calibration curve, then flash frozen and lyophilized.

In FIG. 4H depicting the spectrographs of serum post isolated experiment compared with control serum and pure polymer, PLA was detected in blood (specifically the serum), as shown by the peak in the magnified region (an example of one rat out of 5 tested for the 1750 cm⁻¹ peak ratio). It should be noted that at the end of the isolated loops experiments, there were no visible polymeric particles in the isolated loop (isolated loop seemed deflated). In addition, no polymeric nanoparticles were detected in the isolated loops washouts. Moreover, the isolated loops GI section was also tested for polymeric material (specifically PLA) absorption and no traces of PLA were found in all tested isolated loops. Blood samples were also tested at 1 hour, 2 hours, 3 hours, and 4 hours, in addition to the 5 hour time points presented below. However, only the 5 hour time points are presented due to the fact that only at these time points was the polymer detected, while at all other time points nothing was detected. Lack of PLA detection may be due to PLA being below detection level or that it has not yet reached the bloodstream).

The magnified region in FIG. 4H shows a peak indicative of PLA absorption into blood (1750 cm⁻¹) at 5 hours into isolated loop experiment (the same is true for the 1084 and 1188 cm⁻¹ peaks).

### Measuring and Calculating PLA Content in Dry Serum Samples

To calculate the percentage of PLA in the spectrograph sample, first the zero-hour (serving as the control, before polymer administration) blood samples were analyzed. Below presented in Tables 3 and 4 are the absolute values of the peaks for rats tested for the zero-hour time point and for the 5 h time point, respectively. Values presented are post-baseline and normalization. The produced sample pellets were kept under lyophilization until they were analyzed in order to minimize the water vapors background readings.

Tables 3 and 4 present the peak ratios recorded for the 5 tested rats at zero time point (before injection, Table 3), and the 5 hour time point (post injection to isolated loop, Table 4).

TABLE 3
Zero-hour peak ratios used for calculations. Peak ratios of PLA-
associated peaks at 1750, 1084, and 1188 cm\hat{ }−1 to the serum
peak at 1650 cm\hat{ }−1 before polymer administration.

| Zero hour time | | | |
|---|---|---|---|
| point peak ratios | 1750 cm\hat{ }−1 | 1084 cm\hat{ }−1 | 1188 cm\hat{ }−1 |
| Rat #1 | 0.08119 | 0.08065 | 0.11347 |
| Rat #2 | 0.05040 | 0.07787 | 0.10425 |
| Rat #3 | 0.08610 | 0.12210 | 0.16688 |
| Rat #4 | 0.07872 | 0.07824 | 0.11947 |
| Rat #5 | 0.15857 | 0.17330 | 0.13583 |

TABLE 4
Five-hour peak ratios used for calculations. Peak ratios used by
the per-rat calculation sheets for the 5-hour blood-serum samples.
These are the values that connect to the worked example and final
uptake calculation path

| 5 h dry serum peak | | | |
|---|---|---|---|
| ratios per rat | 1750 cm\hat{ }−1 | 1084 cm\hat{ }−1 | 1188 cm\hat{ }−1 |
| Rat #1 | 0.10700 | 0.08963 | 0.14578 |
| Rat #2 | 0.07349 | 0.07998 | 0.12866 |
| Rat #3 | 0.07852 | 0.08176 | 0.12767 |
| Rat #4 | 0.06935 | 0.08493 | 0.12874 |
| Rat #5 | 0.17850 | 0.24770 | 0.17580 |

Rat #1 is discussed below as an example for calculating systemic uptake from measured and calculated PLA content in dry serum. First, the peaks deltas of ratios for rat #1 at zero-time point (control) were subtracted from their respective 5 h peak ratios post isolated loop administration after baseline correction and normalization were performed on the spectrographs. Then, the subtracted peak ratios are plugged to the calibration curves created with known percentage of PLA in dry serum (FIGS. 4B-4G, see example below). Table 5 represents the peak ratios achieved for rat #1 before (5 h time point, Table 4) and after subtraction of the control peak (zero time point, Table 3) and the resulting concentration using the calibration curve in FIG. 4B. Table 6 represents the same as Table 5 but for the divided calibration curves.

TABLE 5
Rat #1 recorded peak ratios at the three chosen peaks of 1750, 1084,
and 1188 cm⁻¹ (all compared to dry serum peak at 1650 cm⁻¹) and the
resulting percentage in serum based on the subtraction calibration curves.

| Peak ratio to 1650 cm⁻¹ | 1750 cm⁻¹ | 1084 cm⁻¹ | 1188 cm⁻¹ |
|---|---|---|---|
| Control (zero hour) | 0.08119 | 0.08065 | 0.11347 |
| 5 h Sample | 0.10700 | 0.08963 | 0.14578 |
| Delta | 0.02581 | 0.00898 | 0.03231 |
| Calculated % | 2.56 | 3.10 | 4.55 |

TABLE 6
Rat #1 recorded peak ratios at the three chosen peaks of 1750, 1084,
and 1188 cm⁻¹ (all compared to dry serum peak at 1650 cm⁻¹) and the
resulting percentage in serum based division calibration curves.

| Peak ratio to 1650 cm⁻¹ | 1750 cm⁻¹ | 1084 cm⁻¹ | 1188 cm⁻¹ |
|---|---|---|---|
| Control (zero hour) | 0.08119 | 0.08065 | 0.11347 |
| 5 h Sample | 0.10700 | 0.08963 | 0.14578 |
| Delta | 1.3179 | 1.1113 | 1.2847 |
| Calculated % | 1.63 | 3.78 | 3.65 |

As can be seen from the three other subtraction calibration curves, the average percentage of PLA in dry serum was 3.4% 1.03% (mean±standard deviation), while from the divided calibration curves the average was 3.0%±1.2% (mean±SD). These numbers agree well with one another. However, if the concentration is too low (below detection level), another option to quantify the uptake is by spiking the sample with a known amount of PLA. Once this number is obtained, further calculations can be made to translate it into bioavailability as described below.

Determining PLA % using the 1750 cm⁻¹ Cal. Curve (resulting in 2.56%, FIG. 4B):

Zero time (control) point: PLA peak value (1750 cm⁻¹): 0.08119. Dry serum Peak value (since in all recorded spectrographs the 1650 cm⁻¹ serum peak was always the largest once, after normalization it becomes 1): 1.00. Hence the PLA: dry serum peak ratio is: 0.08119.

For the 5 h time point: PLA peak value (1750 cm⁻¹): 0.1070, dry serum peak value: 1.00, hence PLA: dry serum peak ratio is:0.1070.

Then, the zero time point peak ratio is subtracted from 5 h peak ratio resulting in: 0.1070-0.08119=0.02581.
PLA in blood Calibration Curve Equation (an example of 1750 cm⁻¹ subtracted calibration curve):

$$\begin{matrix}Y ( Peak ratios delta ) = 0.0127 \times ( concentration ) - 0.00675 ( {R}^{2} = 0. 0.99514 ) \\ Thus , for 0.02581 peak ratio , x = \frac{0.02581 + 0.00675}{0.0127} = 2.56 \% ( \% of PLA in a blood sample )\end{matrix}$$

Calculating whole blood volume of rats based on their mass

First, the rat's dosage and weight were recorded (see Table 7 below). The rat weight is then used to calculate the blood volume (briefly, for an obese rat the ratio of blood volume vs the weight of the rat is 3.4±0.2 mL per 100 g of rat weight while for regular rats the more “traditional’ ratio of 5.7±0.2 mL per 100 g of rat weight. (Ye, International Journal of Obesity 2009, 33, 606; doi:10.1038/ijo.2009.39). Then the whole blood volume is translated into serum dry mass. In the case of Rat #1 example:

$$Rat ( female ) weight : 367 g . , Total blood volume = 5.7 * ( \frac{367}{100} ) mL = 20.9 mL .$$

Translating Whole Blood Volume into Dry Serum Mass

As these data were not available in the literature, experiments were performed in order to calculate the dry mass of serum in whole blood. In order to achieve this purpose, a SD rat was sacrificed and its blood was divided into 5 vials, each containing 1 mL of whole blood. Then the blood was allowed to clot and the serum was separated (using a centrifuge as described above for the blood samples). Then, the serum was separated, flash freezed and lyophilized. After lyophilization, the dry serum mass was weighed. The results indicated that the dry mass of plasma per 1 mL of whole blood is 42±7.3 mg of serum dry mass per mL whole blood. Again using rat #1 example:

Total mass of dry serum per 1 mL of blood: 42±7.3 mg of dry serum/mL of whole blood. Hence, the total mass of dry serum for this rat is: 20.9 mL*42 mg/mL=878±151 mg

### Calculating Total Mass of PLA in Rats Blood

Obtaining the dry mass of the serum enables the calculation of the total mass of dry serum in order to apply the PLA percentage in dry serum. Using that total mass will enable to calculate how much of it is PLA. Continuing with rat #1 example:

The total mass of dry serum for this rat is: 878±151 mg, PLA content in dry serum was 3.4% or 3.0% (subtracted and divided calibration curves averages, respectively). Hence the total PLA found in blood was: 878*0.034=29.8 mg and 878*0.03=26.3 mg.

### Calculating the Percentage Uptake

In this example, 110.2 mg of PLA NPs were administered to rat #1 in 1 mL of PBS was administered, of which only 91 mg were actually administered (leftover in the syringe, spillage etc.). Systemic uptake is defined as the percentage of the administered dosage reaching the blood circulation. Hence:

Hence, percent of PLA uptake (subtracted calibration curve, 3.4%):

$$\frac{Bioavailable PLA}{Total PLA injected} \times 100 \% = \frac{29.8 \pm 8.1 mg}{91 mg} \times 100 = 32.8 \pm 7.3 \%$$

Hence, percent of PLA uptake (divided calibration curve, 3.0%):

$$\frac{Bioavailable PLA}{Total PLA injected} \times 100 \% = \frac{26.3 \pm 8.1 mg}{91 mg} \times 100 = 28.9 \pm 7.3 \%$$

Table 7 presents all the calculations and additionally recorded data from all the tested rats. All calculations were performed as detailed above for the example for Rat #1.

TABLE 7
Uptake calculation from the percentage in the dry serum sample.

| | | | | | Total | Total | |
|---|---|---|---|---|---|---|---|
| | | | Not | Actual | Blood | Plasma | |
| | Weight | Dosage | used | dosage | volume | mass | Uptake |
| | (g) | (mg) | (mg) | (mg) | (mL)* | (mg)*** | (%) |
| Rat #1 | 367 | 110.2 | 19.2 | 91 | 20.9 | 878.3 | 32.8 |
| (F) | | | | | | | |
| Rat #2 | 270 | 110.3 | 33.2 | 77.1 | 15 | 646.2 | 24.4 |
| (F) | | | | | | | |
| Rat #3 | 450 | 110.4 | 43.9 | 66.5 | 26 | 1077.0 | 0.0 |
| (M) | | | | | | | |
| Rat #4 | 510 | 110.8 | 35.8 | 75 | 29 | 1220.6 | 37.6 |
| (M) | | | | | | | |
| Rat #5 | 766 | 84.6 | 0 | 84.6 | 26 | 1093.5 | 70.4 |
| (M) | | | | | | | |

Although all six calibration curves could be used (FIGS. 4B-4G), the calculation using the “subtracted” calibration curves (FIGS. 4B-4D) were found to be the most accurate and reproducible.

Overall, five rats were evaluated, one of which gave zero amount of systemic uptake and the remaining four that showed systemic blood uptake of 24.4% to 70.4%. The calculations to determine the uptake (%) values shown in Table 7 for each rat were made employing the data in Table 3 and Table 4 for each rat utilizing all three wavelengths.

We claim:

1. Polymeric particles comprising an active agent encapsulated therein,
wherein the polymeric particles comprise one or more polymers and have a zeta potential between −10 mV and −80 mV and a diameter between 100 nm and 5000 nm, inclusive, and
wherein the zeta potential is measured in deionized water at room temperature and pH of between about 5 and about 7.4,
wherein the polymeric particles do not contain chemically bound poly(ethylene glycol) on their surface,
wherein when the polymeric particles are formed from polylactic acid, the polymeric particles comprise polylactic acid with a molecular weight between 2 kDa and 10 kDa, inclusive,
wherein the polymeric particles show systemic uptake between 10% and 80% in a mammal, as measured using Fourier Transform Infrared spectroscopy wherein the polymeric particles comprise a moiety on the surface of the polymeric particles that imparts a negative zeta potential to the polymeric particles, wherein the moiety is bonded to (i) at least one of the one or more polymers or (ii) the active agent encapsulated therein, and
wherein the moiety that imparts a negative charge is selected from the group consisting of carboxylic acids, protonated sulfates, protonated sulfonates, protonated phosphates, singly- or doubly protonated phosphonates, and singly- or doubly protonated hydroxamate, carboxylates, sulfates, sulfonates, singly- or doubly deprotonated phosphate, singly- or doubly deprotonated phosphonate, and hydroxamate.

2. The polymeric particles of claim 1, wherein at least one of the one or more polymers (i) is incorporated in a polymeric matrix that forms the polymeric particles or (ii) is coated on the surface of the polymeric particles.

3. The polymeric particles of claim 1, wherein the size of the polymeric particles is between 100 nm and 800 nm or between 900 nm and 2000 nm.

4. The polymeric particles of claim 1, further comprising anionic surfactants, peptides, lipids, amino acids, salts, or combinations thereof,
wherein the anionic surfactants, peptides, lipids, amino acids, salts, or combinations thereof, constitute between about 0.0001% wt/wt and about 5% wt/wt of the polymeric particles.

5. The polymeric particles of claim 1, wherein the active agent is selected from the group consisting of small molecules, proteins, polypeptides, peptides, carbohydrates, nucleic acids, glycoproteins, lipids, antibodies/antigens, and combinations thereof.

6. The polymeric particles of claim 1, wherein the active agent is glucagon-like peptide-1 (GLP-1) or a truncated biologically active portion thereof or an analog thereof, and wherein the polymeric particle does not contain PAA (poly-adipic acid), PLGA (poly-lactic-co-glycolic acid), or PLA (poly-lactic acid) as the sole polymer forming the polymeric particle.

7. The polymeric particles of claim 1, wherein the one or more polymers are poly(lactic acid).

8. The polymeric particles of claim 2, wherein at least one of the one or more polymers does not dissolve in water within one hour at a pH between 6 and 7, inclusive, at room temperature.

9. The polymeric particles of claim 2, wherein at least one of the one or more polymers has a molecular weight between 1.5 kDa and 300 kDa, inclusive, and when the polymeric particles are formed from polylactic acid, the polymeric particles contain a polylactic acid with a molecular weight between 2 kDa and 10 kDa.

10. The polymeric particles of claim 2, wherein the one or more polymers comprise a blend of a low molecular weight polymer having a molecular weight between 2 kDa and 20 kDa, and a high molecular weight polymer having a molecular weight between 21 kDa and 300 kDa, and when the low molecular weight polymer is polylactic acid, the polymeric particles contain polylactic acid with a molecular weight between 2 kDa and 10 kDa.

11. The polymeric particles of claim 5, having a ratio of the low molecular polymer to the high molecular polymer between 30% wt/wt and 90% wt/wt, inclusive.

12. The polymeric particles of claim 2, wherein:
(i) the one or more polymers are selected from the group consisting of polyesters; poly(hydroxyacids); polyhydroxyalkanoates; polyanhydrides; hydrophobic polypeptides; polyacetals, polycyanoacrylates, polyketals, polyhydroxyvalerates, polyalkylene oxalates, polyalkylene succinates, mixtures, and copolymers thereof,
(ii) at least one of the one or more polymers is bioadhesive and has a bioadhesion force of about 500 mN/cm² or greater, or
(iii) the size of the polymeric particles is between 100 nm and 800 nm or between 900 nm and 2000 nm.

13. The polymeric particles of claim 1, further comprising anionic surfactants, peptides, lipids, amino acids, salts, or combinations thereof,
wherein the anionic surfactants, peptides, lipids, amino acids, salts, or combinations thereof, constitute between about 0.0001% wt/wt and about 5% wt/wt of the polymeric particles.

14. The polymeric particles of claim 10, wherein the anionic surfactant is petroleum sulfonate, naphthalenesulfonate, olefin sulfonate, an alkyl sulfate, sulfated natural oil, sulfated fat, sulfated ester, a sulfated alkanolamide, a sulfated alkylphenol, a sulfated alkylphenol ethoxylate, laurate, lauryl ether sulfate, lauryl sulfate, decyl sulfate, octyl sulfate, a alkylbenzene sulfonate, or a combination thereof.

15. The polymeric particles of claim 1, wherein the active agent is selected from the group consisting of small molecules, proteins, polypeptides, peptides, carbohydrates, nucleic acids, glycoproteins, lipids, antibodies/antigens, and combinations thereof.

16. The polymeric particles of claim 1, wherein the active agent is glucagon-like peptide-1 (GLP-1) or a truncated biologically active portion thereof or an analog thereof, and wherein the polymeric particle does not contain PAA (poly-adipic acid), PLGA (poly-lactic-co-glycolic acid), or PLA (poly-lactic acid) as the sole polymer forming the polymeric particle.

17. A formulation comprising the polymeric particles of claim 1, and a pharmaceutically acceptable carrier.

18. A method for use of the formulation of claim 17 for treatment of a human or an animal body by therapy, comprising administering the formulation to the human or the animal.

19. The method of claim 18, wherein the method comprises orally administering the formulation to the human or the animal.

20. The polymeric particles of claim 7, wherein the one or more polymers are selected from the group consisting of poly(caprolactone), poly(lactic acid), poly(glycolic acid), poly(lactic acid-co-glycolic acid), poly(3-hydroxybutyrate), poly(4-hydroxybutyrate), poly(fumaric-co-sebacic acid), polysebacic acid, polyfumaric acid, hydrophobic polypeptides; polyacetals, polycyanoacrylates, polyketals, polyhydroxyvalerates, polyalkylene oxalates, polyalkylene succinates, mixtures, and copolymers thereof.


SEQUENCE LISTING (10 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 39 aa, Heloderma suspectum
HGEGTFTSDLSKQMEEEAVRLFIEWLKNGGPSSGAPPPS

SEQ ID NO: 5 - protein, 31 aa, synthetic construct
  SITE 20: note = C-16 - C-16 fatty acid (palmitic acid) attached with a glutamic acid spacer on the lysine residue at position 20
HAEGTFTSDVSSYLEGQAAKEFIAWLVRGRG

SEQ ID NO: 6 - protein, 44 aa, synthetic construct
HGEGTFTSDLSKQMEEEAVRLFIEWLKNGGPSSGAPPSKKKKKK

SEQ ID NO: 7 - protein, 645 aa, synthetic construct
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 31 aa, synthetic construct
  SITE 2: note = semaglutide - Xaa = alpha-aminoisobutyric acid
  SITE 20: note = semaglutide - Lys20 is acylated with C-18 stearic diacid (AEEAc-AEEAc-?-Glu-17-carboxyheptadecanoyl)
HXEGTFTSDVSSYLEGQAAKEFIAWLVRGRG

SEQ ID NO: 10 - protein, 30 aa, synthetic construct
  SITE 2: note = Taspoglutide - Xaa = 2-methylalanine
  SITE 29: note = Taspoglutide - Xaa = 2-methylalanine
  SITE 30: note = Taspoglutide - Xaa = L-argininamide
HXEGTFTSDVSSYLEGQAAKEFIAWLVKXX